(19) United States
(12) Patent Application Publication
(10) Pub. No.: US 2023/0298711 A1
(43) Pub. Date: Sep. 21, 2023

(54) Systems and Methods for Addressing Incontinence

(71) Applicant: Kimberly-Clark Worldwide, Inc., Neenah, WI (US)

(72) Inventors: Jonathan D. Boulos, Appleton, WI (US); Sweta Kannan, Elmhurst, IL (US); Sara G. Leonard, Neenah, WI (US); Nicolas Martens, Neenah, WI (US); Marcille Faye Ruman, Neenawh, WI (US)

(21) Appl. No.: 17/923,951

(22) PCT Filed: Jun. 15, 2021
(86) PCT No.: PCT/US2021/037326

§ 371 Date: Nov. 8, 2022

Related U.S. Application Data
(60) Provisional application No. 63/039,205, filed on Jun. 15, 2020.

(51) Int. Cl. G16H 10/60

(52) U.S. Cl. CPC G16H 10/60

(57) ABSTRACT

The present disclosure provides systems and methods for logging incontinence information. The method can include detecting, by one or more computing devices, a user input describing incontinence information; updating, by the one or more computing devices and in response to detecting the user input, a private log based at least in part on the incontinence information; determining, by the one or more computing devices and after updating the private log, a performance metric based at least in part on comparing the private log with a current plan; and providing, by the one or more computing devices, a progress notification based at least in part on the performance metric.

### CROSS-REFERENCE TO RELATED APPLICATION

The present application claims filing benefit of U.S. Provisional Patent Application Ser. No. 63/039,205 having a filing date of Jun. 15, 2020, and which is incorporated herein by reference in its entirety.

### FIELD

The present disclosure relates generally to digital applications including interactive user interfaces for detecting user interactions. More particularly, the present disclosure relates to computing systems including instructions for executing a digital application for addressing incontinence via digital application.

### BACKGROUND

Current incontinence solutions are generally marketed towards older men and women; however, many younger users, particularly women suffer from bladder leaks but may not consider themselves as incontinent. Stigmas around incontinence and other factors, such as a belief that incontinence products are not discrete, may lead women to hide bladder issues rather than finding treatment and/or support, or using the correct incontinence protection.

Currently, digital applications such as bladder diaries, are available for various operating systems, however, these applications are mainly designed and recommended by doctors, leading them to exhibit a very clinical approach. For instance, existing approaches ask users to create a diary of their incontinence without creating concrete solutions for improving incontinence episodes, which leads to limited user engagement and low follow-through.

Needed still in the art are digital applications that can engage women struggling with bladder leaks on multiple levels. For instance, in one aspect, it would be beneficial to provide an application for improving bladder leaks that improves engagement and follow-through of a user. In a further aspect, it would be advantageous to provide an application for improving bladder leaks that creates customized content based upon user inputs to improve engagement.

### SUMMARY

Aspects and advantages of embodiments of the present disclosure will be set forth in part in the following description, or can be learned from the description, or can be learned through practice of the embodiments.

According to one aspect of the present disclosure, a computer-implemented method for logging incontinence information. The method can include detecting, by one or more computing devices, a user input describing incontinence information; updating, by the one or more computing devices and in response to detecting the user input, a private log based at least in part on the incontinence information; determining, by the one or more computing devices and after updating the private log, a performance metric based at least in part on comparing the private log with a current plan; and providing, by the one or more computing devices, a progress notification based at least in part on the performance metric.

According to another aspect of the present disclosure, a system for logging incontinence information. The system can include one or more processors and at least one tangible, non-transitory computer-readable medium that stores instructions that, when executed by the at least one processor, cause the one or more processor to perform operations. The operations can include detecting, by one or more computing devices, a user input describing incontinence information; updating, by the one or more computing devices and in response to detecting the user input, a private log based at least in part on the incontinence information; determining, by the one or more computing devices and after updating the private log, a performance metric based at least in part on comparing the private log with a current plan; and providing, by the one or more computing devices, a progress notification based at least in part on the performance metric.

According to another aspect of the present disclosure one or more non-transitory computer-readable media that collectively store instructions for a digital application for incontinence. The instructions can be for performing operations including detecting, by one or more computing devices, a user input describing incontinence information; updating, by the one or more computing devices and in response to detecting the user input, a private log based at least in part on the incontinence information; determining, by the one or more computing devices and after updating the private log, a performance metric based at least in part on comparing the private log with a current plan; and providing, by the one or more computing devices, a progress notification based at least in part on the performance metric.

### BRIEF DESCRIPTION OF THE DRAWINGS

Detailed discussion of embodiments directed to one of ordinary skill in the art is set forth in the specification, which makes reference to the appended figures, in which:

FIG. 1 illustrates an example of an application manager that can be provided for display by a digital application according to an aspect of the present disclosure.

FIG. 2 illustrates selecting an avatar having one or more features that can be provided for display by a digital application according to an aspect of the present disclosure.

FIG. 3 illustrates further examples of selecting an avatar including selecting one or more design and/or personality attributes according to implementations of an aspect according to the present disclosure.

FIG. 4 illustrates design and personality attributes of avatars according to an aspect of the present disclosure, such as: shape, color, eyes, mouth, and personality.

FIG. 5 illustrates an example interactive user interface for display on a digital application depicting different avatars, a prompt, and interface elements configured to receive one or more user interactions according to aspects of the present disclosure.

FIG. 6 illustrates another example interactive user interface for display on a digital application according to an aspect of the present disclosure. The interface can provide for display a guide, a prompt, and interface elements configured to receive one or more user interactions according to implementations of an aspect of the present disclosure.

FIG. 7 is a simplified schematic illustration of sections of a digital application depicting an onboarding section that transitions to a homepage section including three subsections: community, closet, and your journey according to an aspect of the present disclosure.

FIG. 8A is a simplified schematic illustration of an application structure for an example learning plan that can be provided by a digital application according to an aspect of the present disclosure.

FIG. 8B is a simplified schematic illustration of an example application structure for assigning a new learning plan that can be provided by a digital application according to an aspect of the present disclosure.

FIG. 8C is a simplified schematic illustration of an example application structure for reviewing a new plan that can be provided by a digital application according to an aspect of the present disclosure.

FIG. 8D is a simplified schematic illustration of an example application structure for reviewing a completed learning plan and generating an end of plan assessment according to an aspect of the present disclosure.

FIG. 9A is a simplified schematic illustration of an example structure for logging flows (e.g., incontinence information) according to an aspect of the present disclosure.

FIG. 9B is a simplified schematic illustration of an example structure for Kegel flows according to an aspect of the present disclosure.

FIG. 10A is a simplified schematic illustration of an aspect of a digital application that depicts an onboarding section of the digital application according to an aspect of the present disclosure.

FIG. 10B is a simplified schematic illustration of further aspects of onboarding such as a tutorial according to an aspect of the present disclosure.

FIG. 11 is a simplified schematic illustration of another aspect of a digital application that depicts a community section of the digital application according to implementations of an aspect of the present disclosure.

FIG. 12 is a simplified schematic illustration of another aspect of a digital application that depicts a journey section of the digital application according to implementations of the present disclosure.

FIG. 13 is a simplified schematic illustration of another aspect of a digital application that depicts a closet section of the digital application according to implementations of the present disclosure

FIG. 14 is a simplified schematic illustration of example journeys for different incontinence (Inco) types, including a stress plan, an urge plan, and a combination (combo) plan, according to an aspect of the present disclosure.

FIG. 15 illustrates aspects of application systems for inclusion in digital applications according to an aspect of the present disclosure.

FIG. 16 illustrates an example avatar for a plan evolution system according to aspects of the present disclosure.

FIG. 17 illustrates an avatar associated with a coin system for inclusion in a digital application according to an aspect of the present disclosure.

FIG. 18 illustrates successive avatars for respective plans or levels of a plan of a pal progress system according to aspects of the present disclosure

FIG. 19A illustrates an example progress summary or report card according to aspects of the present disclosure.

FIG. 19B illustrates an example daily summary display according to aspect of the present disclosure.

FIG. 20 illustrates an exercise customization model according to aspects of the present disclosure

FIG. 21 illustrates a computing system for performing incontinence logging according to an aspect of the present disclosure.

### DETAILED DESCRIPTION

Generally, the present disclosure is directed to a digital application for helping reduce or better control the frequency and/or severity of incontinence. The digital application can include a combination of features to promote regular interaction and tracking of bladder events. For example, the digital application can provide encouragement through promotion and/or tracking of progress. The digital application can also provide empowerment by suggesting plans or exercises based on a private user log while giving the user authority to select a current plan. As users interact more with the application, features become unlocked to visually demonstrate progress that may be slower and/or more difficult for the user to detect.

One example implementation according to the present disclosure can include a computer-implemented method for incontinence logging. The method can include providing, by one or more computing devices (e.g., a smartphone, tablet, etc.), an interactive user interface for display on one of the computing devices. The interactive user interface can include one or more interface elements configured to detect user interactions with the interface elements. The method can also include detecting a user input that describes an entry with respect to at least one of the interface elements for logging incontinence information in a private log stored as part of the digital application. For example, the user input can include an interaction with the interactive user interface such as clicking, tapping, or otherwise selecting a user interface element. Additionally or alternatively, the user input can include a dataset descriptive of incontinence information that can be received by the digital application as the entry or that may be processed to generate the entry. For example, sensor data (e.g., voice input or dictation, image data, and/or other data generated by a device) may be received by the digital application (e.g., by transmitting from a secondary computing device such as a home assistant to the computing device running the digital application, or directly by the computing device running the digital application). This sensor day can undergo processing at the secondary computing device or the computing device running the digital application to generate the entry (e.g., by converting the audio to text) that can be used to update the private log.

Based at least in part on the entry, the method can include updating the private log. After updating the private log, the method can include determining a performance metric based at least in part on comparing the private log to a current plan. In some implementations, the digital application can use the progress metric to generate a progress notification and provide for display a system notification that includes the progress notification. Such progress notifications can be provided on periodic basis (e.g., daily, weekly, monthly, etc.) to encourage the user. Additional notifications can be provided to prompt the user to log performance of activities, reports of events (e.g., bladder events), and the like. Thus, the digital application can promote user engagement and provide the user with encouragement to help the user progress on their plan.

Aspects of logging incontinence information can include detecting a user input directed to different interface elements. For instance, different interface elements may include graphics representing a function of the interface element. For example, the interactive user interface can include one or more diary elements configured to record incontinence information. The interactive user interface can also include an interface element for accessing a personal avatar. The personal avatar can be displayed on a separate interface environment that includes one or more avatar manipulation interface elements for creating and/or modifying the personal avatar. Additionally or alternatively, the interactive user interface can include an interface element for accessing a public board. The public board can be displayed on a separate interface environment that includes one or more forum interface elements for posting content to the public board. Thus, the interactive user interface can include interface elements that are configured to detect a user interaction. Upon detecting the user interaction, the digital application can modify the user interface to display new interactive elements that are configured to perform different operations.

In some implementations, providing for display the interactive user interface can be based at least in part on a current plan associated with a user account. The current plan can have one or more defined goals for logging certain incontinence information, such as bathroom trips, liquid intake, leakage, and/or bladder exercises (e.g., Kegels). As such the interactive user interface can also include imagery displaying aspects of the current plan.

Aspects of associating a learning plan as the current plan can include determining a set of one or more learning plans from a plurality of learning plans based on an event, such as include registering a new user and/or detecting that a user account has no logging history. For example, to gain familiarity with the interactive user interface and/or other features of the digital application, the learning plans can include an onboarding plan that can be the same for all users. The onboarding plan can include setting tasks or goals that can be accessed through the interactive user interface. After completing a plan (e.g., the onboarding plan), example implementations can include instructions to provide a new plan (e.g., one of the plurality of learning plans) for display in the user interface. Prior to providing the new plan for display, some example implementations may first provide a recap to review entries received by the digital application. The recap can include questions, data, statistics, and/or other information.

Providing the new plan can include accessing a plurality of learning plans and providing one or more learning plans based at least in part on a log of user history. Each of the learning plans can be differentiated based on a difficulty level, a target incontinence issue, or both. For instance, a user account that has been using the digital application for several weeks may only be provided only high difficulty learning plans. The learning plans can also be accompanied by text or other visual imagery providing information related to the learning plan. Based on a user interaction to select one of the learning plans, said learning plan can be assigned as the current plan.

For example, a method for logging incontinence information can also include providing for display on the interactive user interface, data describing one or more learning plans each including one or more training exercises. Implementations can detect a user input describing a selection of at least one of the one or more learning plans, and based on the selection, assign said at least one of the one or more learning plans as the current plan. Several non-limiting examples of training exercises can include: a video, an audio, a game, and a text.

Aspects of the game training exercise can include tracking an interaction with the interactive user interface, providing for display on the interactive user interface a description of a task and updating and based at least in part on the interaction the description of the task. For tracking incontinence, example tasks can include performing a Kegel and/or a bladder holding event.

In particular, example implementations of the present disclosure seek to engage users through multiple interactive features, such as creating and/or modifying a personal avatar, providing access to a community of other users, providing tangible incentives through coins that users can deploy in various ways including unlocking additional custom features that may be used to update the community and/or the avatar, and tracking regular goals through the use of learning plans. The community aspect can encourage users through interaction with other users experiencing similar issues. One or more of these interactive features can be accessed through the interactive user interface by detecting user interactions.

For some implementations, the current plan can include a goal for using some of all of the features over a given time period. For example, the current plan may include a goal for detecting a user interaction with an avatar element, detecting a user interaction with a community element, or both. Additionally or alternatively, the current plan may include a goal for logging one or more user interactions with diary elements for logging information related to incontinence. For example, the goal may include logging such information daily, at least five days per week, weekly, or the like. If a user does not achieve the goal within the time period (e.g., day, three days, week, etc.), then the method can include providing a notification for display such as a notification reiterating the goal.

Aspects of providing a notification can include providing a notification for display via the interactive user interface and/or providing data descriptive of the notification to a computing device (e.g., via an API) for display by the computing device. Generally, notifications may be sent via one or more different routes. For example, notifications can be provided through other computing devices in communication with the digital application. The digital application can generate a notification and determine one or more communication routes (e.g., web-based notifications, SMS, email, smart assistant, voice assistant, and/or chat bot) for providing the notification. Some notifications may be provided based on a user progress for the current plan and/or other achievements that are detected by the system. These notifications can be accompanied by items or gifts that can be accessed through the user interface. For example, some notifications can include credits, coins, or the like that can be used to buy items from a purchase interface (e.g., closet). In this manner, the digital application can engage users to continue working on plans to improve their incontinence symptoms. An additional method for providing notifications can include push notifications. Such push notification can include a link (e.g., deep link) into the application for the user to provide input. As additional examples, notifications can be web based, provided via test message (SMS), e-mail, interaction with a smart assistant, voice assistant (e.g., as an auditory notification), haptic feedback, and/or “chat bot.”

Some examples of achievements can also include self-progress metrics. For instance, certain implementations can include instructions for determining a self-progress metric, based at least in part on comparing the private log on a first day to the private log on a second day that occurs after the first day. As one example, the number of leaks on the day one of logging bladder events can be compared to the number of leads on day seven to determine a difference (e.g., increase, decrease, or no change) in the number of leaks and/or the amount/quantity of leaks. Using this information, the digital application can provide for display on the user interface data describing the self-progress metric such as a graph. Other activities and interactions can result in rewards as well, such as participation in the community, completing profile data, filling out a survey, or other activities as identified by the application administrator.

Based on user progress, which can include the self-progress metric and/or the progress metric, implementations can update a rewards account. The rewards account can be used to purchase items from the closet that can be used to modify some or a portion of the interactive user interface. Items for purchase can include virtual accessories (e.g., clothing, jewelry, etc.) for the user's avatar, virtual pets for the user's avatar, and/or virtual accessories (e.g., clothing, jewelry, etc.) for the virtual pets. Such accessories can be in limited supply (e.g., a limited number produced) and/or available for a limited time (e.g., a fall-themed accessory may only be available during the fall months or the like). As additional examples, the rewards account balance can be used purchase and/or applied for a discount on physical goods that can be shipped to the user's address. The user's rewards account balance can be used to enter into sweepstakes, drawings, or the like. Conversely, in some embodiments, the user can exchange currency (e.g., U.S. dollars, Euros, cryptocurrency, etc.) for rewards points.

In come implementations, other activities and/or interactions can result in receiving rewards and/or updating the rewards account. Some examples of these activities and/or interactions can include participation in the community (e.g., by receiving data descriptive of generating a post in the community and/or providing support by interacting with messages posted to the community), completing profile data, or other activities as identified by the application administrator (e.g., some examples of these activities are disclosed in the Drawings).

For some implementations, detecting a user interaction with one of the interface elements (e.g., a diary element) can include generating a set of one or more response options. As an example, detecting a user interaction with a liquid element can generate response options that can include a description (e.g., how much liquid was consumed, what type of liquid, and/or what time of day) as well as a response input area. The response input area can be configured to detect a user input (e.g., one cup) so that updating the private log is based at least in part on the user input(s) for the response options. In some implementations, the response option may include a prompt to confirm that the user input(s) provided for the response options are correct.

To track user interactions, some implementations can include registering a user account associated with a unique user id. The unique user id can be associated with logging information such as information included in the private log. In this manner, different user accounts can be created that each may have different progress levels. Thus, the interactive user interface provided for display may also depend in part on the unique user id associated with the user account.

In some implementations, one or more machine-learned models can be leveraged. Data can be aggregated from different users to provide a global data set, which can be used to train the machine-learned model(s). The unique user id can be used to collect incontinence information from one or more user accounts in a substantially anonymous manner. Some example implementations can include aggregating the information from private logs associated with different unique user ids to create a global dataset. The global dataset can be included as a portion (e.g., some or all) of a training dataset for training a global machine-learned model. Alternatively or additionally, some example implementations can include aggregating the information form private logs associated with one unique user id to create a personal dataset. The personal dataset can be included as a portion (e.g., some or all) of a training dataset for training a personal machine-learned model. Thus, personal data from multiple users can be used to train the global machine-learned model(s) and/or personal machine-learned model(s).

In certain implementations, transfer learning techniques may be employed. As one example, a machine-learned model can be pre-trained for a general task and/or a task that is related to treating incontinence. Example tasks can include personal assistant functions, personal trainer functions, food tracking/dieting functions, emotional counseling functions, or the like. The pre-trained machine-learned model can be trained using complied incontinence data, for example to produce the global model.

In some implementations, the personal machine-learned model and/or the global machine-learned model can be used to generate an output based on receiving at least a portion of the private log associated with a user account. The output can include a confidence or other metric for determining a set one or more learning plans from the plurality of learning plans to provide for display on a user interface. In this manner, the machine-learned models can be used to determine which learning plans may provide the greater benefit to a user account based on user logging history.

One aspect of generating the training dataset for the global machine-learned model and/or the personal machine-learned model can include labeling the training data. For instance, example implementations of the present disclosure can include providing for display a notification that includes a question and/or one or more response fields. The question or response fields can be used to clarify a user interaction and/or obtain additional information related to incontinence events such as information stored in the private log, user satisfaction with the current plan, user satisfaction with progress, or a combination of these. Some example questions are depicted in the Drawings as notifications (e.g., “how are you feeling today”, “was today leak free”, etc.). User responses to these questions, however, do not necessarily need to be included as updates to the private log. Instead, in some implementations, these user responses can be used to label the global dataset and/or the personal dataset, by associating the user responses as labels that can be applied for performing supervised learning techniques without updating the private log based on these responses. The training dataset can include also include sensor data (e.g., from accelerometers, pressure sensors, capacitive sensors, volatile organic compound sensors, and the like).

Another example aspect of the present disclosure can include integration and/or communication with other devices, applications and/or products. In some implementations, these devices, applications, and/or products can be limited to incontinence uses. Alternatively, certain implementations can include integration and/or communication with various devices, applications, and/or products that are generally directed to health including diet journal (e.g., including logs of volumes of liquid consumed), exercise sensors (e.g., pedometers), temperature and/or oxygen level sensors, etc. For instance, the notifications provided for display by the digital application can include data received from other incontinence devices or products that a user account is currently using. Examples of such data include (questions, activity details, Kegel exercises, information detected by sensors, and/or other similar data). Based on the data received from these devices, applications, and products, the private log may be updated to include description of the specific device, application, or product in addition or alternatively to data acquired by said specific device, application, or product. This information may be used to determine suggested incontinence products that can be provided to users of the digital application. As one example, a machine-learned model (e.g., the personal machine-learned model and/or global machine-learned model) can be trained to suggest an incontinence product using a training dataset that includes labels having information related to the incontinence device and/or products associated with one or more user accounts.

Example devices can include electrical stimulation devices and movement sensing/detection. Electrical stimulation devices can include muscle and/or nerve stimulation devices. Muscle stimulation devices can be configured to induce muscle contractions through applying electrical energy to the muscles, such as pelvic floor muscles. Repeated muscle contractions through electrical stimulation can be used to strengthen the targeted muscles (e.g., pelvic floor muscles) to treat incontinence. Electrical stimulation devices that are configured to stimulate nerves can improve nerve function to treat incontinence. Such electrical stimulation devices can electrically stimulate nerves associated with pelvic floor control and/or bladder function. As one example, an electrical stimulation device can be configured to stimulate the saphenous nerve located in the leg, which has been found to improve bladder control.

Aspects of the present disclosure are directed to interfacing directly with such devices and/or interfacing with third-party digital applications configured to operate such devices. The digital application may include functionality to communicate directly with external products and/or devices, for example, using an application programming interface (API) of the device developer and/or via an API provided with and/or included with the digital application. The API can help third part application and/or device developers to more easily provide feature integration that can improve user experience using the digital application. The API can leverage GraphQL and REST in some embodiments.

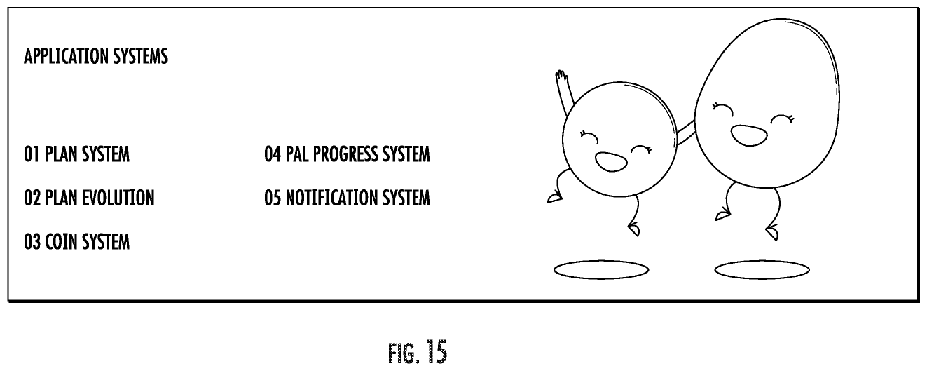

As examples, a learning and/or training program (e.g., customized for the user) can include a nerve and/or muscle stimulation regimen, which can be defined by parameters for using the nerve and/or muscle stimulation device. Example parameters can include frequency, time of day, duration, and/or settings specific to the stimulation device (e.g., intensity, pulse pattern, breaks between electrical pulses, and the like). For instance, a treatment regimen can include one or more repetitions including (1) an applied electrical treatment to cause a muscle contraction for X seconds (e.g., one second to 60 seconds or longer) followed by (2) a period break without muscle contraction for Y seconds (e.g., one second to 60 seconds or longer). In some embodiments, the electrical stimulation treatment regiments can correspond with the “games” described below with reference to FIGS. 15-17.

In some embodiments, the digital application can automatically administer the stimulation according to the stimulation regimen. The digital application can receive a user input indicating that the device is correctly place and/or that the user is ready to begin the regimen. In response to receiving such as user input, the digital application can automatically administer the regimen by causing the nerve and/or muscle stimulation device to apply electrical pulses according to the regimen. Thus, the digital application can apply nerve and/or muscle stimulation treatment via one or more devices.

Similarly, such devices can collect data regarding the regimen and/or the user's reaction to the regimen (e.g., with or without applying an electrical treatment). For instance, a device may be configured to sense a strength, duration, etc. of a muscle contraction (e.g., in response to an applied electrical treatment and/or in response to a prompt instructing the user to tense a muscle, such as a pelvic floor muscle). and/or collect user data (e.g., exercise data) via the device(s) (e.g., according to a treatment regimen prescribed and/or generated for the user by the digital application, such as a custom treatment regimen).

In some embodiments, the digital application can facilitate communication (e.g., wireless or wired) communication between the user's computing device and the stimulation device. In other embodiments, the digital application may communicate (e.g., through an API) with a third-party application configured to control the nerve and/or muscle stimulation device. For instance, the digital application can transmit data describing the stimulation regimen proscribed by the digital application to the third-party application. The third-party application can communicate instructions to the device to automatically administer electrical treatment according to the digital application.

With reference now to the Figures, example embodiments of the present disclosure will be discussed in further detail.

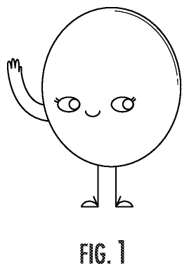

FIG. 1 depicts an example icon/avatar that can be rendered for display by example digital applications of the present disclosure. The digital application can store the example icon as part of system files that may be locally included as part of the application package for download to a local device. For instance, the icon may be encoded in an image format (e.g., JPEG, GIF, MPEG, IMG, PNG, PDF, JSON etc.) that can be stored in memory of the local device. Alternatively, the example icon can be stored as part of a remote computing system (e.g., a server). In this manner, accessing the example icon may include communicating instructions for transmitting image data from the remote computing system to a local device. In some embodiments, the icons/avatars can be designed to help ensure women feel that the avatars represent their personal creative output and aesthetic, while being unrealistic enough to help the women disassociate from the condition.

For implementations of the present disclosure, the icon can act as an application manager that can be provided for display on the interactive interface as part of one or more application sections. For example, the digital application can be divided into one or more application sections such as an onboarding section for training a new user about application systems and or functions, a plans section for tracking user interactions over a time period, and/or a review section for displaying trends based on inputs received by tracking user interactions. In each of these application sections, the icon can be displayed as a form of system or application manager to help guide user interactions. To guide user interactions, the example icon may be provided for display with other imagery such as figures (e.g., shapes, arrows, etc.), animations (e.g., fireworks, cut scenes, etc.), and/or text that can also be accompanied by audio. One example aspect of the icon can include a system defined appearance rather than a custom appearance. In this manner, the icon can be differentiated from other imagery provided by the digital application such as an avatar.

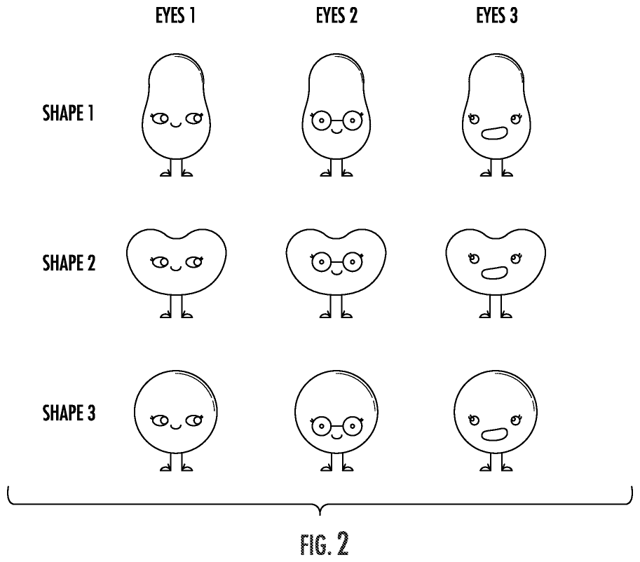

FIG. 2 depicts example avatars that can be rendered for display by example digital applications of the present disclosure. FIG. 2 illustrates variations of the avatars 4 to illustrate that the avatar can include one or more customizable attributes. For instance, FIG. 2 depicts three different types of eyes (eyes 1, eyes 2, and eyes 3) as well as three different types of shapes (shape 1, shape 2 and shape 3). Additional example attributes can include avatar color, hair, hair style, arms, hands, and nails.

Example digital applications according to the present disclosure can include interfaces for a user to create a custom avatar by selecting choices for the one or more customizable attributes. For example, an avatar interface can include one or more avatar interface elements for designating selections for the one or more customizable attributes based on receiving a user interaction with the avatar interface elements. The one or more avatar interface elements can include a predefined set of choices (e.g., shape 1, shape 2, and shape 3), and a user interaction with one of the choices can be used to indicate the choice so that the application detects the choice. Alternatively or additionally, the one or more avatar interface elements can include a customization feature such as a drawing application. Using the customization feature, the user can create their own avatar or attributes by manually drawing a shape, drawing one or more attributes, and/or defining a color (e.g., using an RGB color value scale). The avatars can relate personas and/or illustrations of how the avatar will interact with the user.

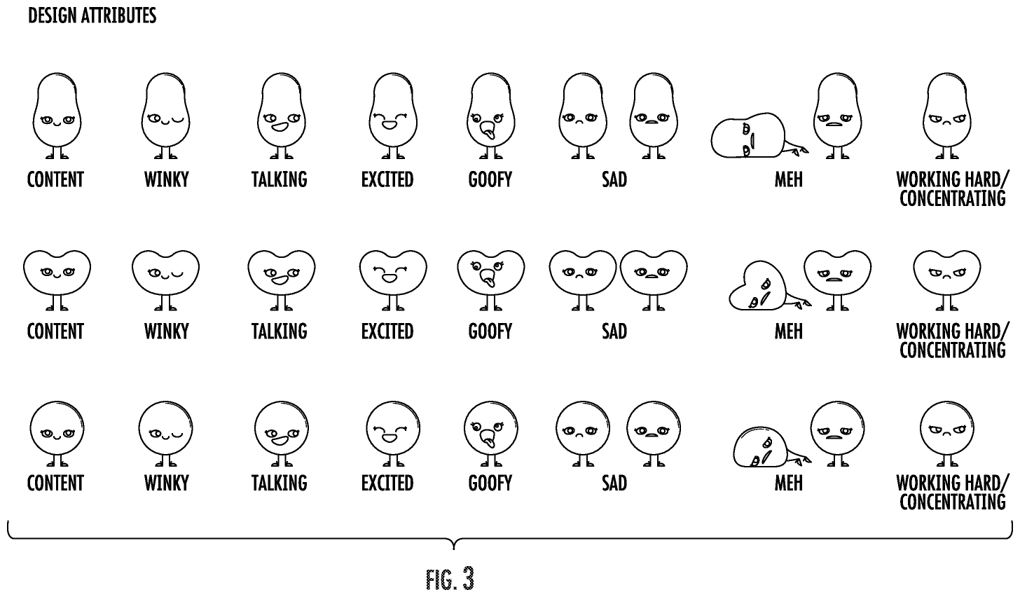

FIG. 3 depicts another aspect of the avatar design according to some implementations of the present disclosure. In FIG. 3, the avatar is depicted to include further design attributes such as a status (e.g., content, winky, talking, excited, goofy, sad, meh, and working hard/concentrating). For some implementations, the status can be included as one of the one or more avatar interface elements that can be designated based on receiving a user interaction with said avatar interface element. Alternatively or additionally, the status can be determined based at least in part on user inputs received by the digital application. As one example for illustration, the status may be determined by the system based on a regularity of detecting user inputs and/or a type of user input with the digital application. For instance, if the digital application has not detected a user input in a timespan (e.g., eight hours, one day, one week, etc.) the application may update the status of the avatar to the sad status, or if the digital application has detected multiple user inputs (e.g., two or more, three or more, five or more, etc.) directed to an application feature such as the community board, the application may update the status of the avatar to the talking status.

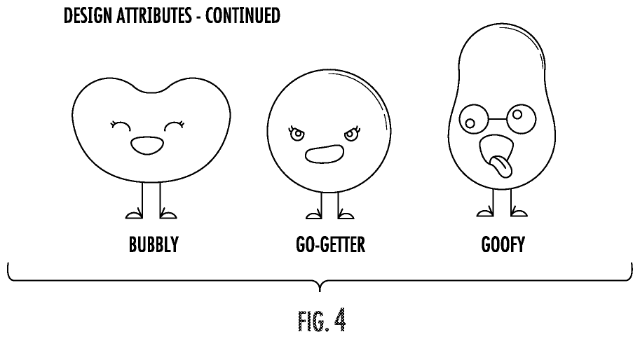

FIG. 4 depicts an example user interface (e.g., a “home screen”) including additional design attributes. These design attributes, such as the avatar status, as provided herein as examples and are not intended to limit the digital application to only including choices for creating an avatar based on the options depicted herein. Rather, digital applications according to the present disclosure can include features (e.g., interfaces) for creating and/or updating an avatar that can be provided for display by the digital application. The digital application can include instructions for accessing one or more files including data directed to the avatar attributes that can be combined to render the avatar based on the user interactions. For instance, the application can access one or more files and apply the files to modify an attribute of the avatar (e.g., the eyes and/or mouth) to depict a status such as bubbly, go-getter, or goofy. The various options can indicate the responsiveness of the avatar to the user's actions, which can make the experience more immersive for the user.

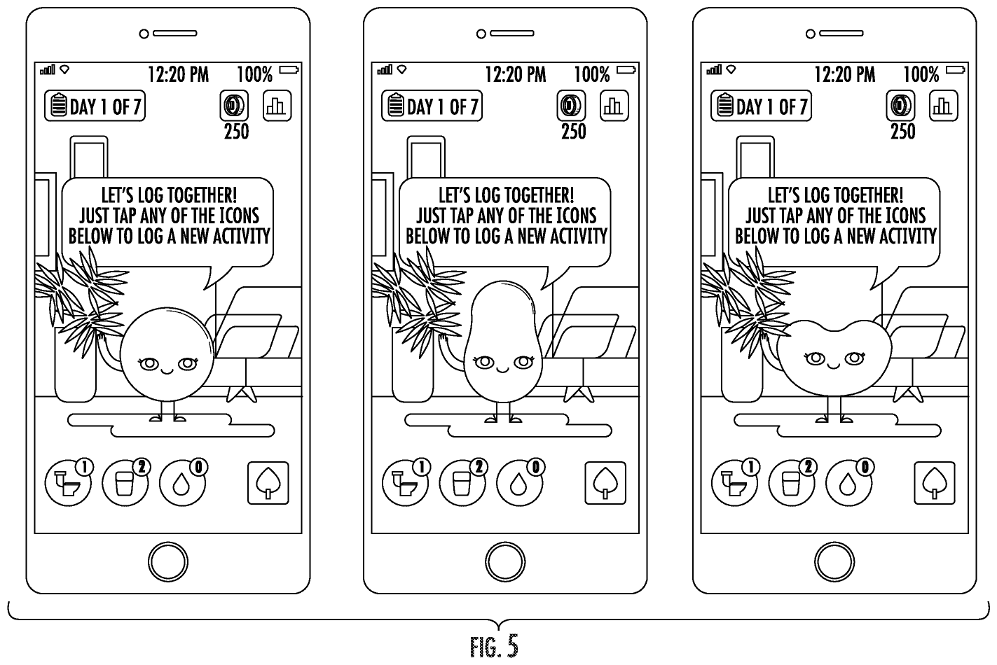

FIG. 5 depicts an example digital application implemented on an example computing device. The example digital application can include instructions for providing an interactive user interface for display on the computing device. The interactive user interface can include one or more interface elements (e.g., buttons) configured to detect a user interaction with the interface element. For instance, a digital application implemented on touch screen devices can be configured to detect a user interaction such as touching an area of the screen depicting the interface element. Alternatively or additionally, the digital application can be configured to detect a user interaction such as a cursor to click on the interface element.

The interactive user interface may also include imagery depicting an avatar, a prompt, and/or a progress status. Additionally, the interactive user interface can be personalized to a user account, such that providing for display the interactive user interface can include providing for display the avatar, the prompt, or the progress status based at least in part on a history of user interactions associated with the user account. For instance, the digital application can store such personalized features by registering a user account associated with a unique user id. In this manner, upon accessing the digital application, the digital application can provide the interactive user interface associated with the unique user id for display, such that the interactive user interface is customized based on prior user interactions that are associated with the user account. To illustrate this feature, FIG. 5 depicts three different interactive user interfaces, each displaying a different avatar.

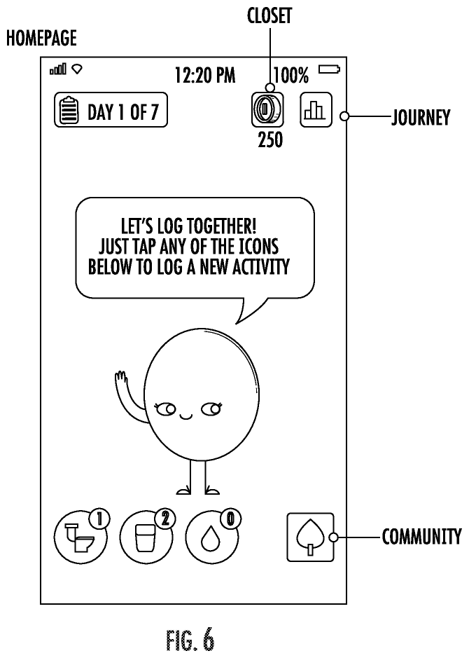

FIG. 6 depicts one example aspect of the interactive user interface showing one or more interface elements that can include a closet that can be used to access a rewards account for purchasing items using an application currency, a journey that can be used to access a private log of incontinence information associated with the interactive user interface, and a community that can be used to access a public board for posting and/or viewing content. Additionally or alternatively, the interactive user interface can include one or more incontinence logging interface elements (e.g., diary elements) that can be associated with incontinence. As shown in the example image, the incontinence logging interface elements can include imagery such as a toilet to indicate bathroom trips, a water glass to indicate liquid intake, and a droplet to indicate leakage. Additional logged data can include age, weight, BMI, heath issues, medications that the user is currently taking and the like. In this manner, inputting incontinence information can be streamlined to focus on certain events based on the interactive elements provided for display.

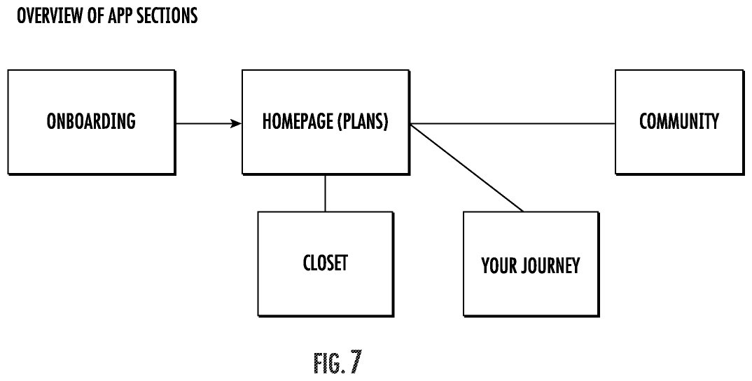

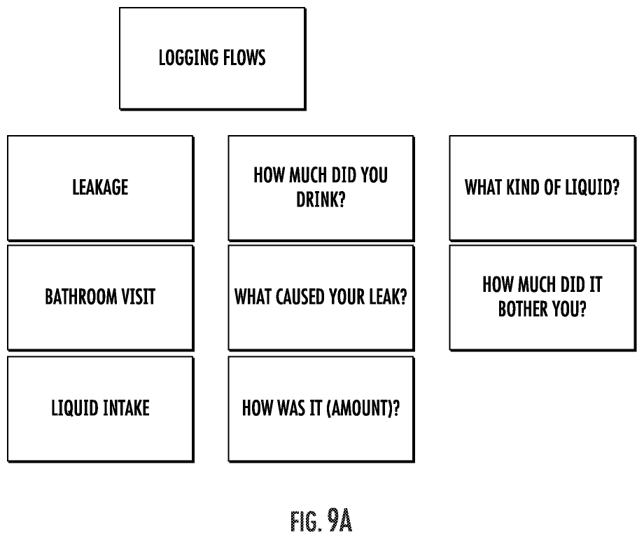

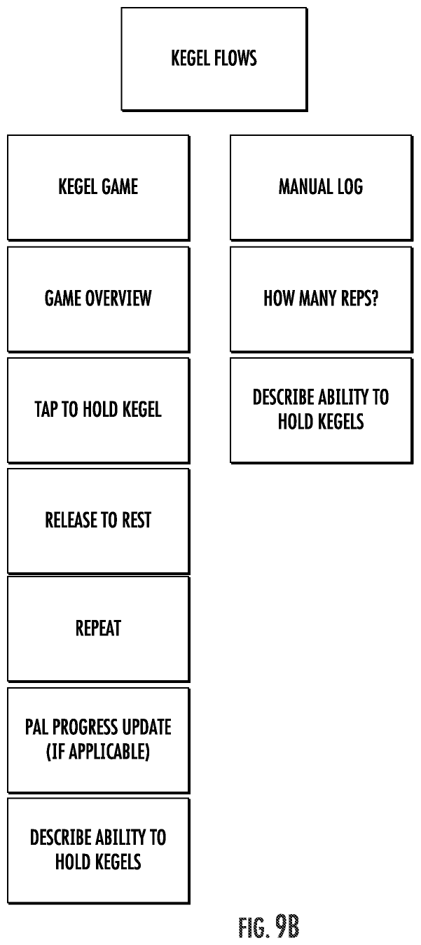

FIG. 7 depicts one example flow displaying an overview of sections included in the digital application (e.g., app sections). A section included in the application can include definitions for what actions a user of the application can make and what actions the system can perform in response. As one example, a digital application according to the present disclosure can include multiple sections such as an onboarding section, a homepage section, a community section, a closet section, and a your journey section. Certain sections may be provided by the application based on a certain event. For instance, when first accessing the application on a new device, or based on a status associated with the application, the application may provide for display an onboarding section. The onboarding section can include a tutorial to explain features of the digital application such as interactive elements on the user interface using prompts. The onboarding section may also include limited interactive features such as only providing interactive elements (e.g., buttons) that are configured to linearly cycle (e.g., forwards or backwards) through a sequence of image frames. Alternatively or additionally, the onboarding section can include an immersive experience by guiding the user to interact with features that can be accessed through the interactive user interface. As one example, in some implementations the onboarding section can include creating the avatar. Another example of the onboarding section is provided with reference to FIG. 9.

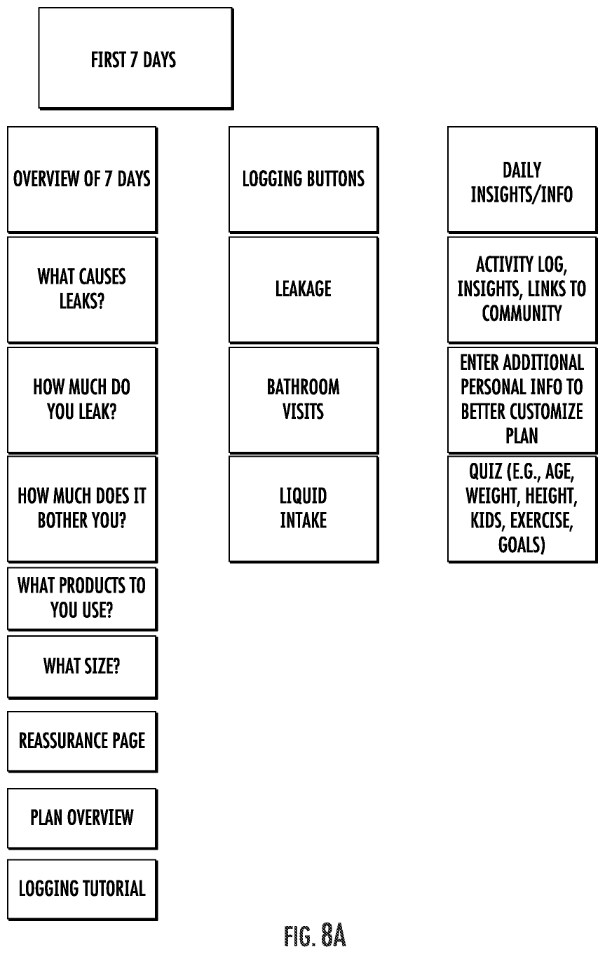

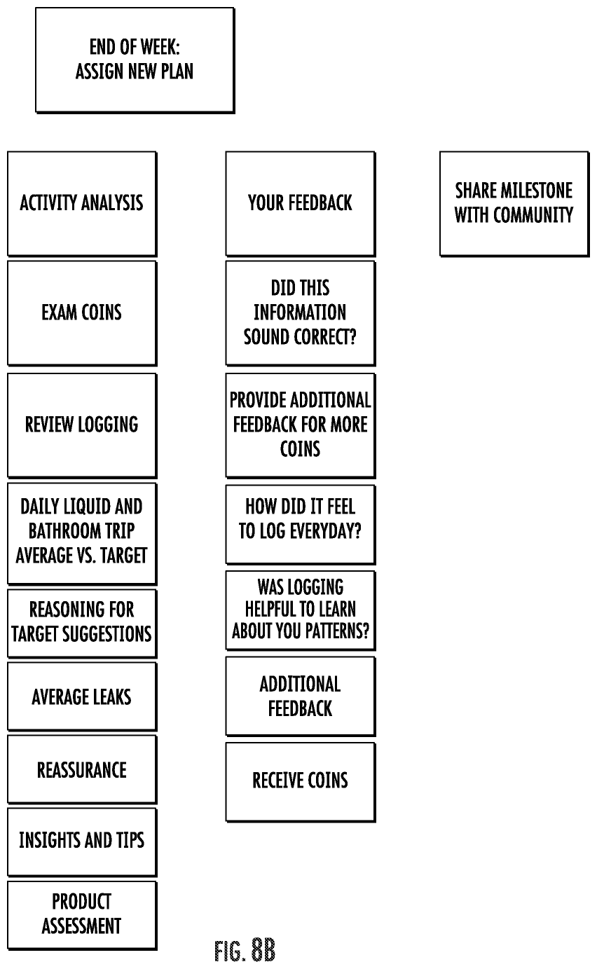

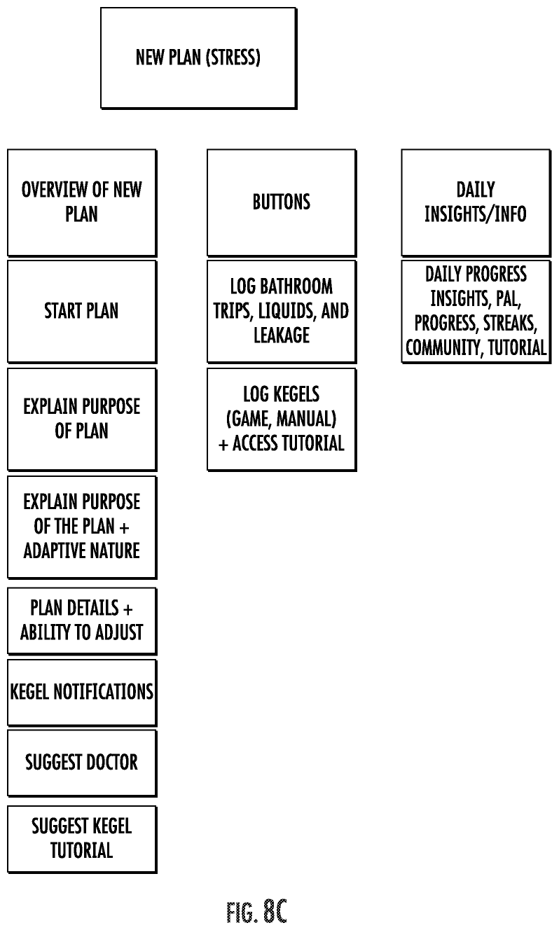

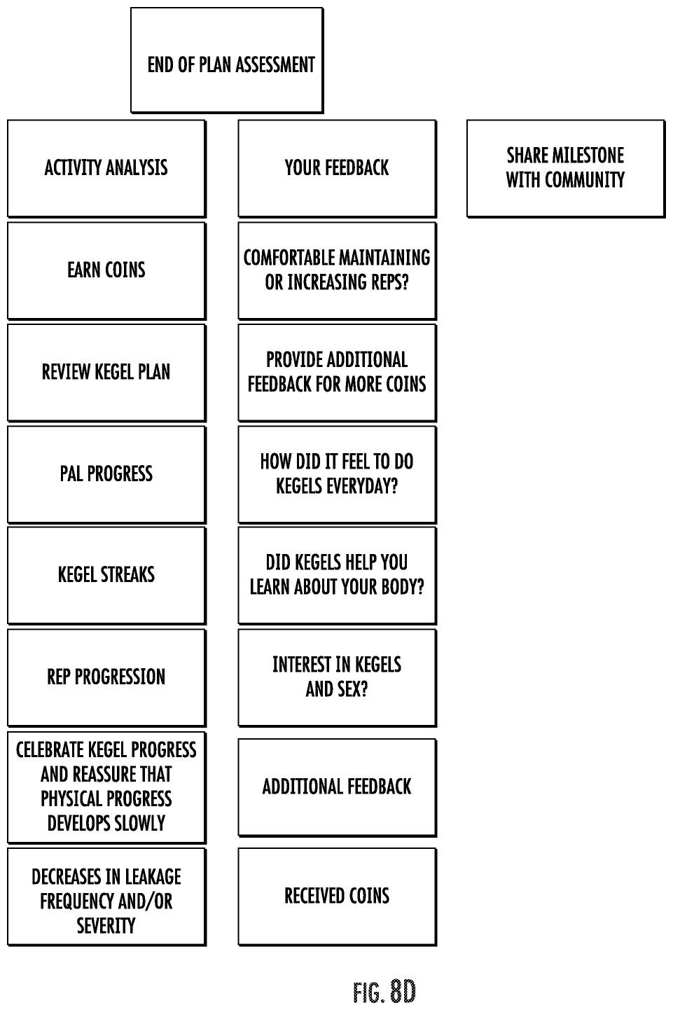

FIG. 8A depicts an example structure for an example learning plan. The structure can provide elements of a learning plan such as example notifications (e.g., what causes leaks?, how much do you leak?, reassurance, an overview of goals, a tutorial for logging incontinence information, etc.) that can be provided for display while the learning plan is active as the current plan. The structure can also include a description of interface elements (e.g., logging buttons) that can detect a user interaction to update a private loge for tracking incontinence information such as leakage, bathroom visits, and/or liquid intake. A further aspect of the structure can include insights or other information that can be provided for display at a regular increment (e.g., every 6 hours, 8 hours, 12 hours, 24 hours, 48 hours, etc.) while the learning plan is active as the current plan.

FIG. 8B depicts an example structure for determining a new plan to assign as the active plan. Aspects of determining the new plan can include reviewing prior activity, providing feedback, and/or sharing events and/or milestones with the community. One aspect of reviewing activity can include one or more of examining coins earned, reviewing logging of incontinence information such as leaks and/or bladder exercises such as Kegels, comparing (e.g., using a graph or other visual representation) daily liquid intake and/or bathroom trips versus a goal or target, providing data to explain goal or target suggestions, providing insights and tips, and providing product assessment. Additionally, for some implementations, prior to starting a new plan, the digital application can request feedback by providing for display one or more notifications to determine a user comfort with the application, while goals of a learning plan, or other related information. To share the milestone of completing a learning plan, the digital application may also provide for display a prompt to share the milestone with the community, which, if a user agrees, can cause the interactive user interface to display a suggested post that can be added to the community based on a user interaction such as selecting a “post” button or other icon for submitting content to the community.

FIG. 8C depicts another example structure for an example learning plan. Similarly to FIG. 8A, the structure can provide elements of the learning plan such as an overview of the new plan, logging buttons, and insights/info to demonstrate user progress. In some implementations, the structure of the plan can be customized based on detecting one or more interactions with data descriptive of elements of the learning plan. For instance, if a user is already familiar with the logging buttons (e.g., diary elements) then the user can skip having the application provide for display information related to the plan overview. As another example, while depicted as “daily” insights/info, it should be understood that a user may customize the plan to display progress at various times or to not include displaying progress while a learning plan is active as the current plan.

FIG. 8D depicts an example structure for an end of plan assessment. The end of plan assessment can be used to provide information on user activity, to request feedback, and to share milestones with the community. Aspects of finishing a learning plan can include earning coins, and/or reviewing progress such as avatar (Pal) progress, Kegel streaks, rep progression, decreases in leakage, etc. It can also include celebrating (e.g., through notifications, animations, or other content that can be provided for display) progress and reassuring that physical progress may develop slowly. Some examples of feedback can include questions, which upon receiving a user response associated with the question (e.g., data responding to the question) may cause the application to generate coins or other tokens that can be provided to update the user account.

FIG. 9A depicts example elements for logging incontinence information (e.g., logging flows) that can include one or more of leakage, bathroom visit(s), and liquid intake. Each of the logging flows can be provided for display as an interface element that can detect a user interaction. In response to the user interaction, some implementations may provide one or more response options. For example, a user interaction with the leakage interface element may cause the digital application to provide for display a follow up question (e.g., how much did you drink?, what kind of liquid?). Additionally or alternatively, a user interaction with the bathroom visit interface element may cause the digital application to provide for display a follow up question (e.g., what caused your leak?, how much did it bother you?). In this manner, some implementations can include logging incontinence information by detecting user interaction and providing a follow up notification for display to clarify further aspects of the incontinence event.

When the user is logging liquid intake, the user interface may provide the user with common serving sizes and/or common beverages from which to select. This may reduce the amount of work for the user to log their liquid intake as compared with other methods, such as typing the serving size and/or beverage type or otherwise selecting the serving size and/or beverage type in a more granular way.

FIG. 9B depicts example elements for performing Kegel exercise. For example, a Kegel game can include reviewing a game overview that can include instructions for performing an interaction with the interactive user interface (e.g., tapping to hold a Kegel, releasing to rest, etc.) The game can also include instructions for performing a number of repetitions and based on game performance determining a progress update such as a pall progress update. Certain implementations can also include method for manually logging Kegels such as entering a number of Kegel reps and/or a description of comfort/ability to hold Kegels.

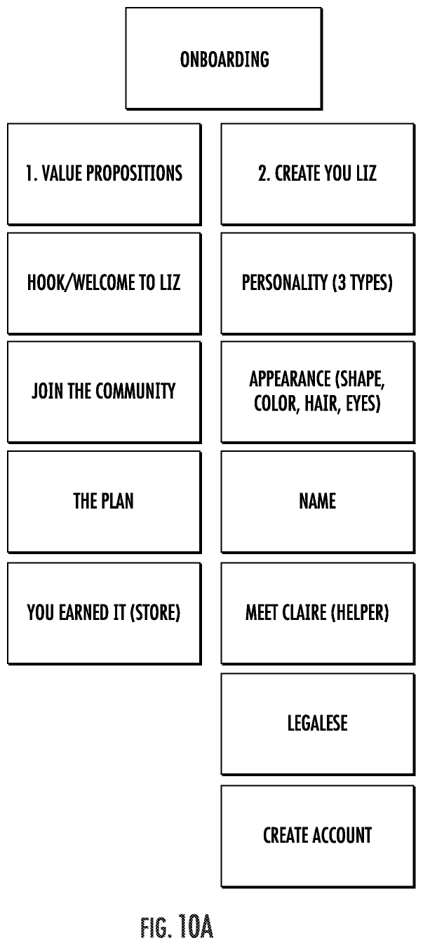

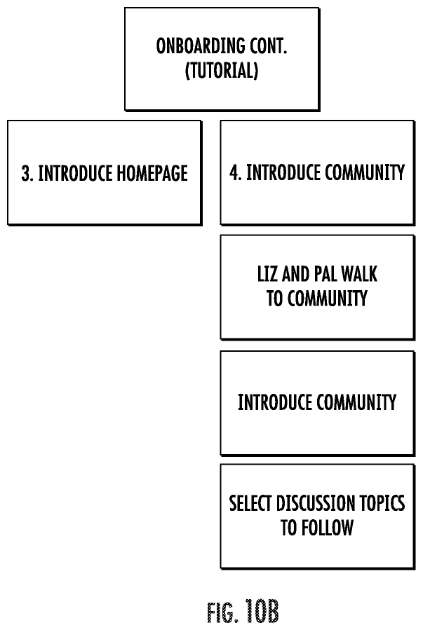

FIGS. 10A and 10B depict an example onboarding section for inclusion in example digital applications. The onboarding section can include providing for display information related to the value of the digital application such as imagery welcoming a user to the platform (Liz), prompts for a user to join the community, data descriptive of the plan or plan(s) the user can access, and a reward (you earned it) that can be used in the digital application. In some implementations, the onboarding section can also include creasing an avatar (your Liz). Creating the avatar can include selecting various attributed according to aspects of the present disclosure such as selecting a personality, which in some implementations may be limited initially to a certain number of different options (e.g., 3 types), selecting an appearance (e.g., choosing shape, color, hair, and/or eyes), and selecting a name. Additional aspects of creating the avatar can include meeting a guide (e.g., Claire) who may be provided for display by the digital application at different times, and displaying other relevant information such as legal terms (legalese) prior to creating an account to register a new, unique user to the digital application.

While FIGS. 10A and 10B illustrate numbered events (value proposition, create your Liz, introduce homepage, and introduce community) it should be understood that these events can occur in any order and that the onboarding section depicted provides one example for onboarding a user to access features of the digital application. For example, in some implementations the onboarding can directly introduce the homepage as illustrated in FIG. 6 using an icon to review features of the digital application such as the community, the journey, and/or the closet. Aspects of introducing the community can include providing for display imagery describing Liz and pal walk to the community, introducing the community, and imagery prompting to select discussion topics in the community to follow.

FIG. 10 depicts an example homepage (plans) section that can be provided for display using a computing system. The homepage can be modified to include different features based on a current plan. For instance, during an initial period (e.g., seven days) the digital application may include a plan for new users. An example plan for new users can include features such as an overview of the seven days; logging buttons for tracking incontinence events such as leakage, bathroom visits, and liquid intake; and daily insights. While a plan is active as the current plan, one or more of the features of the plan can be provided for display at all times or based on an event. For instance, at some point each day, the application may provide for display a prompt including one or more of recent activity such as a log of user inputs, a request to enter additional information, and/or a quiz.

Another example of an event can include completing a current plan. Completing the current plan can include meeting one or more plan goals such as having the plan assigned as the current plan for a certain period of time (e.g., one day, three days, one week, etc.) and/or completing one or more plan exercises (e.g., watching tutorials, playing games, etc.) In general, after completing the current plan, a new plan can be assigned. Additionally, a summary of the completed plan can be provided for display including information such as activity insights (e.g., daily bathroom trips, leakage trends, etc.) that can be based on inputs received by the application from user interactions while the plan was active as the current plan. These insights can also be coupled with prompts requesting additional input and or feedback regarding events logged while the plan was active as the current plan. In some cases, the digital application may also provide a prompt for display. The prompt can include text and or animations to support progress using the application and may also request permission to share the achievement with the community as a public post.

After completing a plan, the digital application may provide for display a suggestion to start a new plan. The suggestion to start a new plan can include presenting one or more new plans on the interactive user interface. At least one of the new plans can then be assigned as the current plan based on receiving a user interaction with the new plans.

In some embodiments, one or more of the plans can be adaptive in nature. For example, the plans can be adjusted based on the adapting/evolving needs, severity, and/or abilities of the user. The plans can further be adjusted based on the user's frequency of interaction, past level of success in plans, and perceived motivation.

Some example aspects of starting the new plan (e.g., assigning the new plan as the current plan) can include providing for display an overview of the new plan that can include: starting the plan, explaining the purpose of the plan, explaining the purpose of the plan including any adaptive features, a summary of plan details such as goals and the ability to adjust or customize said goals, a request for Kegel notifications, suggesting a doctor, and/or suggesting a Kegel tutorial.

The cycle of completing a plan, performing an end of plan assessment, and starting a new plan can be continued to provide further activity insights to users of the digital application, as well as allowing users to continue to earn coins for meeting plan goals. Aspects of the assessment can also include providing feedback about comfort managing incontinence using exercises such as Kegels and/or ability to hold bathroom events. The digital application can include instructions providing certain prompts based on previous information logged by a user account. In this manner, the prompts to provide for display on the interactive user interface can be customized. Additionally or alternatively, certain implementations may include a set sequence of prompts. Aspects of the plan may also determine if the digital application provides customized prompts. For instance, the introductory plan (e.g., first 7 days) may not include customized prompts since the digital application has not accumulated enough logging information to determine whether a prompt should be provided based on the private log associated with a user account.

As previously noted, a new plan may include different interface elements (e.g., buttons) for logging incontinence information. For instance, the new plan may include interface elements for logging bathroom trips, liquids, and leakage in addition to interface elements for logging Kegels (e.g., for tracking Kegels performed during a game or tutorial). Thus, after selecting a new plan, the interactive user interface may be modified to include different interface elements or other features.

The new plan can also include notifications to provide insight and/or encouragement while the new plan is active as the current plan. Some examples of notifications that can be provided for display include, daily progress, insights (e.g., graphs displaying trends), pal progress, streaks (e.g., consecutive days logging incontinence information), community notifications (e.g., updates to topics that an account is following), and tutorials.

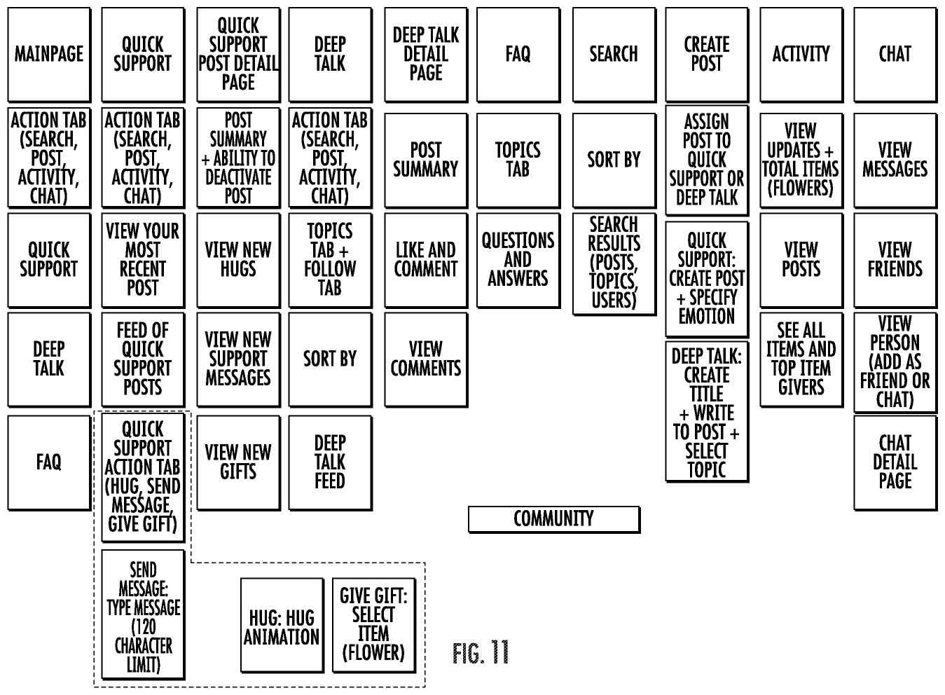

FIG. 11 illustrates an example organization for the community, which can include features such as a main page, FAQ, search, create post, and activity. These features can be used to create a public board that can be used to host content created by users of the digital application. On accessing the community, one or more of the features can be provided for display on the interactive user interface. These features may be organized on a main page as illustrated that can include an action tab (search, post, activity, chat), sort by, support feed, and FAQ. Aspects of the features can include digital representations such as buttons or other interactive elements that are configured to detect a user interaction with the feature. If the feature detects an interaction, the digital application can modify the user interface. For instance, the sort by feature can be used to change an order or items presented on the main page, and the create post feature can be used to present a new interactive window that can overlay a portion of the main page or can be presented in a new window. Additional features can include text boxes that can be used to search for posts, topics, and/or users. Thus, the community can generally act as a forum that can be accessed through the digital application

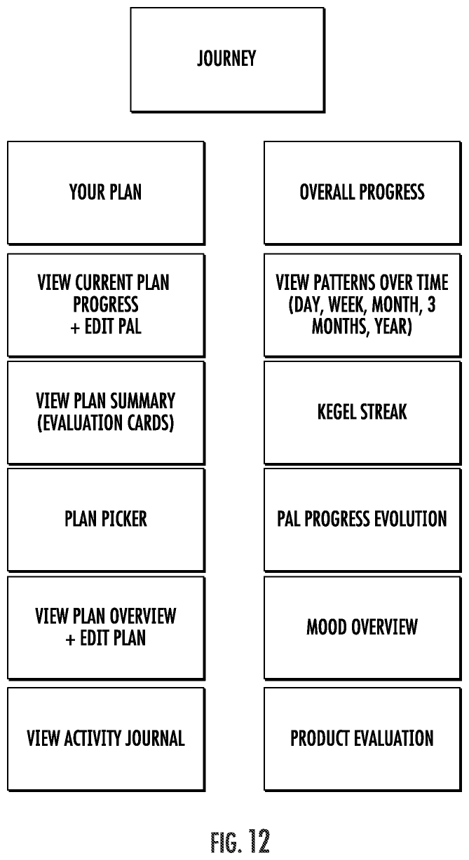

FIG. 12 illustrates an example organization of the private log (e.g., journey). The private log can include a history of prior interactions with features of the digital application. These interactions can be divided into your plan (e.g., a current plan and/or previous completed plans) as well as overall progress that can track a complete history of user logged actions (e.g., interactions with interface elements). The “your plan” section can be used to access information such as viewing and/or editing the current avatar associated with a user account with or without accessing the avatar interface. The avatar can be considered as a virtual friend or pal who is going along with a user during use of the digital application. As such the digital application may modify the avatar to display different features as a user account progresses through different plans or performs other actions using the digital application. Additionally or alternatively, your plan can include a summary of the plan (e.g., current progress in meeting plan goals and/or activity logged while the plan is active as the current plan), a plan picker which can include a summary of other plans offered by the digital application, a plan overview(e.g., a summary of goals or other actions that the plan can track) that in some implementations can be edited, and/or an activity journal which may be limited to only display activity logged while the plan is active as the current plan.

The private log can also keep track of overall progress logged by a user account. The overall progress can be used to view patterns of logging incontinence events over a timespan (e.g., by day, week, month, three months, year, etc.) The overall progress can also keep track of milestones such as a longest Kegel streak (e.g., longest time holding a Kegel, most number of Kegels in a session, days in a row performing Kegel exercises, etc.) Additionally or alternatively, the overall progress can include an evolution displaying progress of the avatar over time, a mood overview, and/or a product evolution.

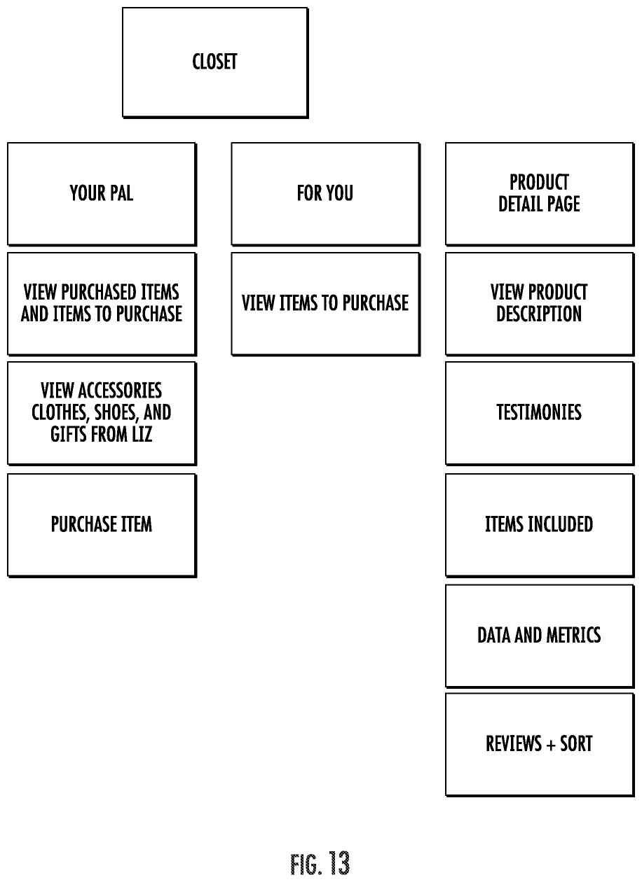

As depicted in FIG. 13, another aspect of an example digital application can include a closet that can be configured to purchase items for modifying the avatar (your pal). The closet can be accessed through the interactive interface and can include providing for display a closet interface that can include an area displaying the avatar, an area for viewing purchased items and items to purchase, an area for viewing accessories, clothes, shoes, and gifts from Liz. A currency (e.g., coins) of the digital application can be used to purchase items from the closet. The currency can be received as a reward for various actions performed by a user account such as performing regular logging of incontinence information or other actions as illustrated in examples depicted in the Drawings.

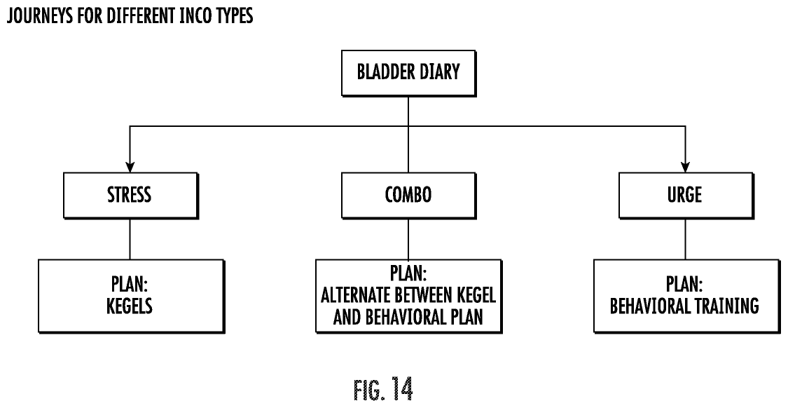

FIG. 14 illustrates an example of journeys for different incontinence (Inco) types. These journey types may be provided for display to a user as part of a current plan and/or based on prior logging events including prior completed plans. The figure depicts how the private log (e.g., a bladder diary) can be used to determine a type of incontinence related to stress, urge, or a combination of both. In this manner, the system can utilize information associated with a user account to determine what plans to provide to a user account and/or provide information to the user account to help select a plan. Example plans can include training goals such as Kegels, behavioral training, or both.

FIG. 15 illustrates three example Kegel games that can be accessed using an example digital application. The Kegel games can have different difficulties based on comfort of a user account with performing Kegels. The Kegel games can also include a description of the game and/or suggestions that can be provided to the user account prior to initiating the game, while the game is active, or both.

Each column of the below table illustrates an example Stress Incontinence Kegel game (SUI) that can be included as part of digital applications for managing incontinence according to an aspect of the present disclosure:

| GAME 1 | GAME 2 | GAME 2 |
|---|---|---|
| (NOVICE) | (INTERMEDIATE) | (MASTER) |
| Hold for 3 sec. | Hold for 5 sec. | Hold for 10 sec. |
| Relax for 3 sec. | Relax for 6 sec. | Relax for 10 sec. |
| Repeat this set 10 times | Repeat this set 10 times | Repeat this set 10 times |
| Play this game 3 times a | Play this game 3 times a day | Play this game 5 times a day |
| day, morning, noon and | | |
| night | | |
| You can do this exercise in | You can do this exercise in a | You can do this exercise in |
| a standing, sitting, or lying | standing, sitting, or lying | a standing, sitting, or lying |
| down positions | down positions | down positions |

Each column of the below table illustrates an example Urgency Incontinence Kegel games (UUI) that can be included as part of digital applications for managing incontinence according to an aspect of the present disclosure:

| GAME 1 | GAME 2 | GAME 2 |
|---|---|---|
| (NOVICE) | (INTERMEDIATE) | (MASTER) |
| When you feel the need to | When you feel the need to | Continue to increase the |
| go, try holding off (contract | go, try holding off (contract | time until you get to 3-4 |
| your pelvic floor muscles | your pelvic floor muscles | hours between bathroom |
| repeatedly) for at least 2-3 | repeatedly) for at least 4 | visits |
| minutes. Then go to the | minutes. Then go to the | |
| bathroom. | bathroom. | |
| Start doing the game in a | Do this for a couple of days | |
| “safe place” i.e., at home. | until you are confident | |
| Do this for a couple of days | | |
| until you are confident | | |

As one example of an example plan for managing incontinence using a mix of Kegel games that can be included as part of a digital application for managing incontinence according to an aspect of the present disclosure can include performing SUI games 1-3 and UUI games 1-3.

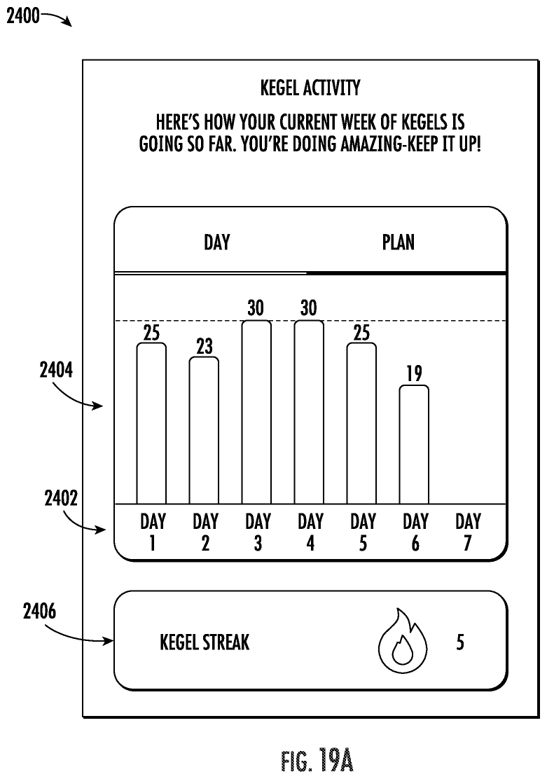

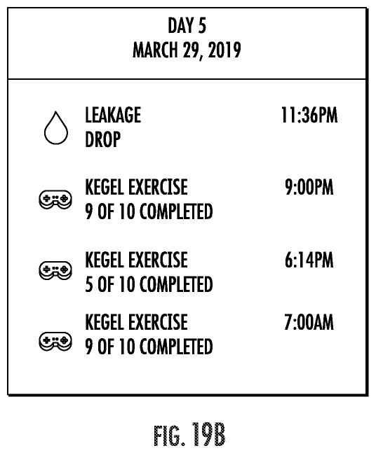

FIG. 15 illustrates an example overview of application systems that can be included in an example digital application. As depicted, the application systems can include a plan system, a plan evolution, a coin system, a pal progress system, and a notification system. FIG. 19 illustrates one example of a plan system. The plan system can keep track of plan goals such as keeping a bladder diary, performing Kegels and other exercises including bladder training. An example aspect of the plan system can include providing personalized plans. In some implementation, all persons who use the digital application can have access to a bladder diary to track incontinence patters. Example implementations can use information included in the bladder diary to determine suggested learning plans. For instance, in one example implementation, a Kegel plan may be suggested if the app notices that pelvic floor muscles could use some strengthening (stress incontinence); and a bladder training plan may be suggested if the app notices frequent trips to the bathroom (urgency incontinence). If the app detects a combination of both (mixed incontinence), the app can cycle through both plans. The intensity and gradual increase in difficulty of each plan is tailored to needs and patterns. Additionally, some implementations can include the option to adjust the learning plan to better fit personal needs such as lifestyle and schedule.

Example aspects of the bladder diary can include a logging period for tracking bathroom trips, liquid, and leakages to identify and understand personal patters. The Kegel plans can include workouts that can be tailored, for example, based on information received by the digital application such as logged incontinence information. The workout can help strengthen pelvic floor muscles by performing the exercises for a period of time. Based on performance and/or comfort level, the exercises can increase and/or decrease in difficulty.

Example aspects of the bladder training plan can include exercises to extend the time in between bathroom trips. These exercises can include breathing games and/or Kegel games. Based on performance and/or comfort level, the exercises can increase and/or decrease in difficulty.

Example text that can be displayed to describe a plan according to aspects of the present disclosure is outlined below.
The Liz app provides you with personalized plans to help you gain control. All women who use the app will start their journey off with a bladder diary to track their patterns. The app will take this information to figure out what plan is best for you.
A Kegel plan may be given to you if the app notices that your pelvic floor muscles could use some strengthening (stress incontinence); and a bladder training plan may be given to you if the app notices that you have frequent trips to the bathroom (urgency incontinence). If you experience a combination of both (mixed incontinence), the app will cycle you through both plans. The intensity and gradual increase in difficulty of each plan is tailored to your needs and patterns. you will always have the option to adjust your plan to bitter fit your lifestyle and schedule.
Throughout your journey you'll be asked to fill out additional bladder diaries so both you and the app can measure if there has been a noticeable decrease in leakage volume and/or frequency. This will help the app understand if it needs to make any adjustments to your future plans.
Bladder diary is a 7 day logging period where you will track your bathroom trips, liquids, and leakages so we can identify and understand your unique patterns. This will help create a framework of plans that are personally crafted to help you gain more control.
Kegel plan: Kegel plans are made to help you gain control at your own pace by strengthening your pelvic floor muscles. You'll get a tailored workout for a period of time that will slowly increase in difficulty until you see significant results. Play Kegel games with your pal to spice up your exercises.
Bladder training plan: Bladder training plans work to gradually extend the time in between your bathroom trips. You'll play breathing games and Kegel games every time you feel the urge to go to the bathroom. Each plan will encourage you to slowly increase the amount of time between your trips.

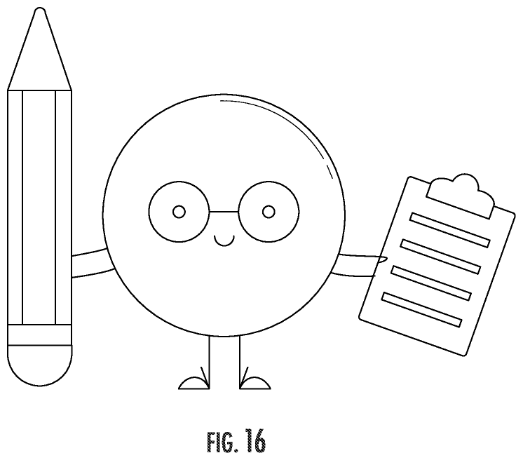

FIG. 16 illustrates an example avatar for a plan evolution system according to aspects of the present disclosure. The digital application can include a type of leveling system for tracking user progress and advancing the difficulty of the plan and/or goals associated with a plan based on user progress.

As one example for illustration, the plan evolution system can include a leveling system of different difficulties (e.g., novice, intermediate, and master). The digital application can determine the appropriate starting level, based on patterns and needs logged in the digital application. One or more of the different levels (difficulties) can be treated as milestones that can result in a system notification being provided for display when a user achieves the milestone.

According to aspects of the present disclosure, a user's plan can change or evolve according to an aspect of the present disclosure. The following is example text that can be displayed for the user.
The plan evolution system is designed to help lay the foundation for how plans can evolve over time; it is not a public-facing naming convention for plans. The Kegel and bladder training plans have a basic leveling system composed of 3 levels: novice, intermediate, and master. The app will determine the appropriate starting point for you based on your patterns and needs. These 3 levels should be treated as milestones. There may need to be additional plans developed for you to graduate from a novice plane to intermediate plan and an intermediate plan to a master plan. These additional plans might be increments of all this plans to create a smoother progression for users. Additional consultation with health professionals will be needed to fully build out the plan systems for Kegel and bladder training, which includes duration of each plan as well.

| | Novice | Intermediate | Master |
|---|---|---|---|
| Kegels | Squeeze: 3 sec. | Squeeze: 6 sec. | Squeeze: 10 sec. |
| | Relax: 3 sec. | Relax: 6 sec. | Relax: 10 sec. |
| | Repeat: 10 times | Repeat: 10 times | Repeat: 10 times |
| | Amount: 3 times | Amount: 3 times a day | Amount: 5 times |
| | a day | Position: Standing or | a day |
| | Position: Standing, | sitting | Position: |
| | sitting, or lying down | | Standing, sitting, |
| | | | or lying down |
| Bladder | Instructions: When you | Instructions: When you feel | Instructions: |
| Training | feel the need to go, try | the need to go, try holding | Continue to |
| | holding off (contract your | off (contract your pelvic | increase the |
| | pelvic floor muscles | floor muscles repeatedly) | time until you |
| | repeatedly) for at least 2- | for at least 4 minutes. Then | get to 3-4 hours |
| | 5 minutes. Then go to the | go to the bathroom. | between |
| | bathroom. Location: | Location: Anywhere, | bathroom visits. |
| | bathroom or home | Amount: a couple of days | location: |
| | Amount: a couple of days | until you are confident | Anywhere |
| | until you are confident | | |

In this example three levels are described (novice, intermediate, master), however it should be understood that more than three levels or less than three levels can be included in the digital application.

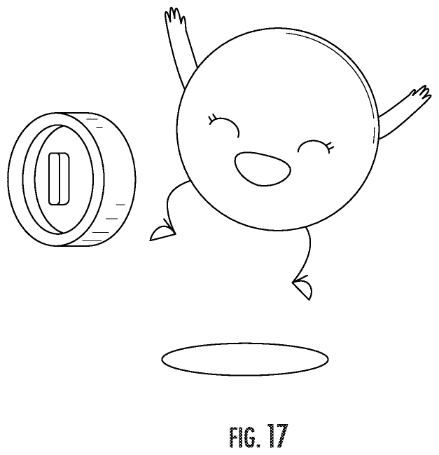

FIG. 17 illustrates an example avatar for a coin system according to aspects of the present disclosure. The coin system can provide a rewards mechanism that updates a user account to receive coins based on user interactions and/or achievements. The coin system can provide different tiers of rewards (e.g., coins) based on activities related to the current plan (e.g., user check-ins, activity (e.g., bladder) logging, accessing tutorials or other content, completing plans, etc.). Alternatively or additionally, the rewards tiers can be based on activities associated with the public board (e.g., community) such as creating a post and/or acknowledging posts associated with other accounts of the digital application. For example, the public board can include a commenting feature such as giving a hug and/or giving flowers to indicate that a user appreciates the post. The commenting feature can include providing a button or other interface element that can be associated with a visual graphic to indicate a flower or hug. When the digital application detects a user account has interacted with a commenting feature, the post can be updated to display the comment and/or the user account can be updated to include the comment as part of the private log (e.g., journey).

As one example for illustration, the coin system can reward user for effort and investment in using the digital application. Users can receive coins for performing activities and the coins can be used to purchase accessories for your pal (avatar) as well as gifts for other users such as community notifications (e.g., flowers) that can be issued as publicly viewed notifications in the community. Different interactions with the application can result in receiving a reward. Examples include logging incontinence information, checking in the application, Pal (avatar) progress, completing notification requiring a user response (e.g., questions and questionnaires), and viewing tutorials.

In some embodiments, the user may have an avatar that is different and distinct from a host or guide character. The guide character can be periodically surfaced to provide the user with guidance through the program in a more personal manner. The guide character can serve as personal coach to lead the user, for example through the tracking and strength training plans. The guide character can be a partner for the user on the journey, for example, even for users who do not actively engage or engage very little in the community aspect of the present disclosure.

The system can identify users that are not particularly engaged with the community. Additionally or alternatively, the system can identify users who try to engage with the community, but are not receiving the feedback and/or support needed from other users within the community. For example, the system can identify characters based on one or more of the following: a frequency at which the user creates posts for the community boards, a frequency at which the user messages other users in the community, a frequency at which other users engage with the user (e.g., by commenting on the user's posts, messaging the user etc.), and/or a frequency or occurrence of the user drafting a post but not actually posting to the community board

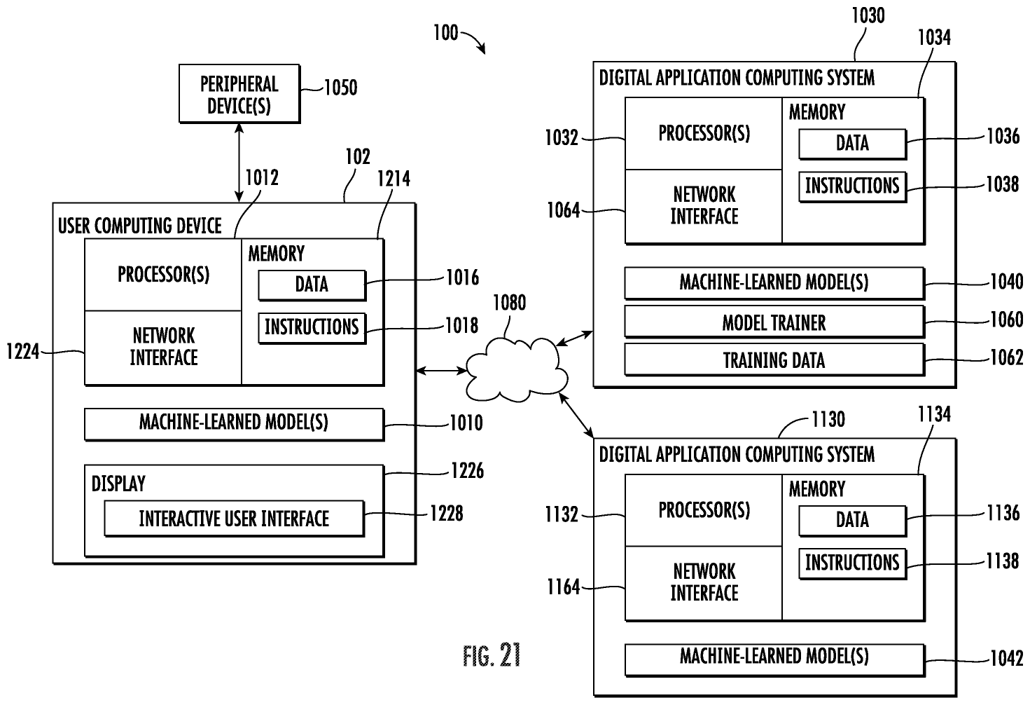

The guide character even be surfaced to offer rewards for such users. Such users may be particularly in need of support and encouragement as the above traits may indicate the user is not receiving sufficient support and encouragement through the community. For example, the guide character can be surfaced to encourage someone who is offering support to others but having trouble with communicating (e.g., in an instance where the community board is primarily or entirely conducted in a language with which they are not fluent). The guide character can reward the user, for example, with coins or special accessories (for the closet). The guide character can also offer “words of encouragement” to the user. This may be accomplished through a combination of artificial intelligence (AI) (e.g., by leveraging one or more machine learned models as described below with reference to FIG. 21) and/or through supervision and/or moderation by another human. The way in which the guide character engages with each user can be customized for each user. As such, the guide character can provide greater support and impact for the users.

Digital currency or coin can be used to purchase items or other rewards by user accounts associated with the digital application. The following text can be displayed to the user to describe the coin system:
The coin system rewards you for all the effort and investment you put into gaining more control and supporting women in the community. You'll get coins for doing activities big and small, and can also purchase more coins in the store. The coins can be used to buy fun accessories for your pal and flowers for other women in the community. The chart below breaks down all the activities that are rewarded with coins, from basic rewards to high rewards.

The following table illustrates example rewards for plan activities and community activities.

| | Plan Activities | Community Activities |
|---|---|---|
| Basic Reward | Logging check-ins | Commenting giving hugs |
| Example: 20 coins | | |
| Medium Reward | Pal progress | Gifting Flowers |
| Example: 50 coins | Streaks (Kegels) | |
| | Longest intervals | |
| | games | |
| High Reward | Questionnaires | Creating a post |
| Example: 100 coins | Tutorials | |
| | Evaluations | |
| | End of plan | |

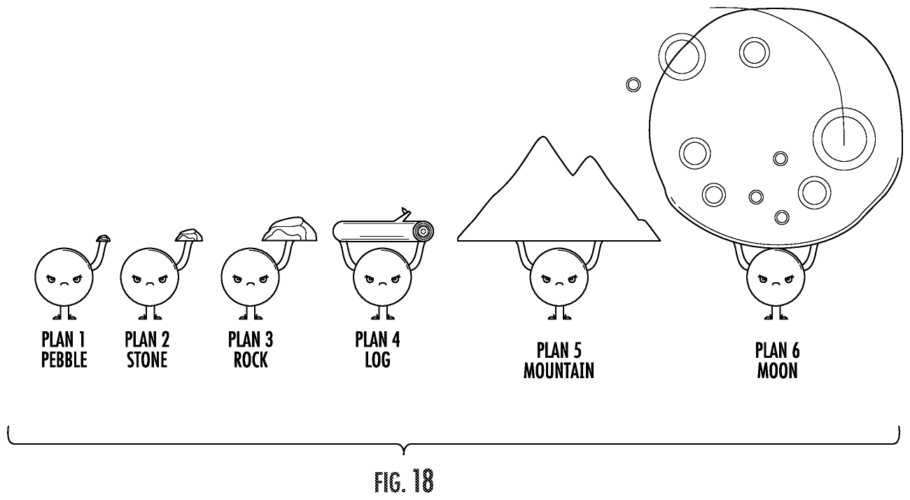

FIG. 18 illustrates successive avatars for respective plans or levels of a plan of a pal progress system according to aspects of the present disclosure. The successive avatars can be used to depict progress metrics that a user of the digital application achieves by modifying the appearance of an avatar/pal that the user can create. The Pal or avatar is a personal feature for each user account and can be used to reflect progress in managing incontinence. While progress may be difficult to interpret using only numbers and/or trends, the pal progress system can provide a visual means for displaying progress.

As one example for illustration, while completing a current plan, the pal can increase the amount of object it can carry by 1 for every 5 activities logged and games played (e.g., bladder diary, Kegel, bladder training). The number and/or size of objects that the pal can hold may be visualized in imagery such as text or pictures.

Further, in some embodiments, the avatar can be animated while the user completes exercises to aid the user in performing the exercise. For example, the avatar can perform a first action (e.g., lift an object and hold it) for a first duration corresponding to a duration of a prescribed Kegel exercise. The animation can inform the user of the required duration and/or emotionally encourage the user during the exercise (e.g., by displaying “almost there, five more seconds). Other exercise parameters, such as intensity, rest duration between reps and/or sets, and the like may be described by the animation to aid the user. As a further example, the avatar can sing a verse of a song, read aloud a passage of a story, or the like while the user refrains from urinating. Once the avatar has completed the verse, story, etc. the user can be instructed to urinate. The user can be provided additional instructions for refraining from urinating. For example, the avatar can instruct the user to sit on the toilet while refraining, stand outside the bathroom while refraining, or the like. The duration of the avatar's song, story, or the like and/or additional instructions associated with refraining from urinating may be increased or otherwise adjusted during the training regimen. For example, the avatar can sing additional verses of a song, a song with longer verses, a longer story, a longer portion of the story, or the like.

The following text can be displayed to the user to describe the pal progress system.
Your pal experiences progress with you by getting stronger throughout your plans. She'll be able to lift a large object after each plan that you complete. It typically takes a few weeks to a couple months for women to notice significant results from Kegel exercises and bladder training. The pal progress system was created to ensure that you feel a sense of progression whenever you use the Liz app. During each plan, your pal can increase the amount of objects it can carry by 1. For every 5 activities logged and games played (bladder diary, Kegel, bladder training). The number of objects your pal can hold is visualized with text. Example Claire can lift 4 logs!

According to some aspects of the present disclosure, a notification system can be used to generate text and/or other notifications that may be personalization, for instance, based on prior interactions with the user interface. The following example text can be displayed for the user to describe the notification system.
Notifications serve as a way for your pal to keep you engaged by communicating with you outside of the app. Tapping on a notification will open the app and bring you directly to its related screen. Below are a list of examples for how notifications can be used. Note: the push notifications will be written in the voice of your pal's personality.

The following table provides example notifications for various activities. The following table also lists which screen may be displayed.

| Activity | Notification | Screen |
|---|---|---|
| Check-in | Hi! How are you feeling | Check-in screen |
| | today? | |
| Logging | I'm a little thirsty! Have we | Liquid logging screen |
| | drank any water recently? | Leakage logging screen |
| | Was today leak free? | Bathroom logging screen |
| | 000 I really have to go! Log | |
| | a bathroom trip with me. | |
| Insights | I have something new and | Insights interaction screen |
| | cool to tell you! | |
| Community | Aww! Someone sent you a | Community post activity |
| | hug/gift/message, let's see | screen |
| | what it is. | Community post |
| | There's a new discussion I | |
| | think you'd like happening in | |
| | Community, wanna check it | |
| | out? | |
| Kegels | If you're ready, it's time to | Kegel game screen |
| | do Kegels in a few minutes | |
| | :) | |
| Closet | Omg! There's some new cute | Closet screen |
| | stuff in the store, let's go | |
| | shopping! | |

The system can include a notification system according to aspects of the present disclosure. The notification system can be customized for each user account based at least in part on features of the avatar (pal) associated with the account. For instance, notifications can be written based on the personality of the avatar (e.g., excited) and/or based on the color of the avatar. In this manner, the notification system can be personalized and less intrusive. Several non-limiting examples of notifications can include providing (e.g., generating a push notification on a device associated with the digital application) text such as notifications depicted in FIG. 23. Notifications may also be provided for display based on a condition, such as accessing a screen of the interactive interface. The screen can include displaying the notification and/or user response elements (e.g., buttons, text boxes, etc.) In some implementations, the notification can only be provided when first accessing the screen, for instance, when the application detects an interactions with a user interface element for displaying the screen (e.g., clicking on the closet interface element on the homepage can bring up the closet screen and display a notification such as the example notification in Table 1.)

TABLE 1
Example Notifications for Display

| | Notification | Screen |
|---|---|---|
| Check-in | Hi! How are you feeling today? | Check-in screen |
| Logging | I'm a little thirsty! Have we drank any water | Liquid logging screen |
| | recently? | Leakage logging screen |
| | Was today leak free? | Bathroom logging screen |
| | Ooo I really have to go! Log a bathroom trip | |
| | with me. | |
| Insights | I have something new and cool to tell you! | Insights interaction screen |
| Community | Aww! Someone sent you a hug/gift/message, | Community post activity |
| | let's see what it is. | screen |
| | There's a new discussion I think you'd like | Community post |
| | happening in community, wanna check it out? | |
| Kegels | If you're ready, it's time to do Kegels in a few | Kegel game screen |
| | minutes :) | |
| Closet | OMG! There's some new cute stuff in the | Closet screen |
| | store, let's go shopping! | |

FIG. 19A illustrates an example progress summary 2400 or report card according to aspects of the present disclosure. A number of activities (e.g., Kegel activities) 2404 performed for each day 2402 can be illustrated in a bar chart 2400 format. An activity streak (“Kegel Streak”) 2406 can be displayed indicating a number of days that the user has performed at least one activity or has performed more than a minimum threshold number of activities as prescribed by the user's plan. However, various other charts, plots, graphics or the like can be employed to provide the user with information about his or her progress, such as completing sufficient activities, having fewer leakage events, or the like.

The progress summary 2400 can be generated and/or surfaced for the user periodically (e.g., at the end of each day, week, month, etc.). Such periodic surfacing of the progress summary 2400 can improve visibility, communication, and drive greater awareness of the user's progress, which can in turn foster greater engagement with the system and/or compliance with the user's training program. Further, at the end of a program, the report can be expanded to include offering for a maintenance program for the user. In some embodiments, the report can include visualizations and/or animations to help the user understand their progress and/or provide a sense of accomplishment.

FIG. 19B illustrates an example daily summary display according to aspect of the present disclosure. Numbers of various activities (e.g., “Kegel exercises”) that the user completed can be displayed along with a time at which the activities were completed. Logged leakage events or other incidents reported by the user can also be displayed along with times at which they were reported to have occurred.

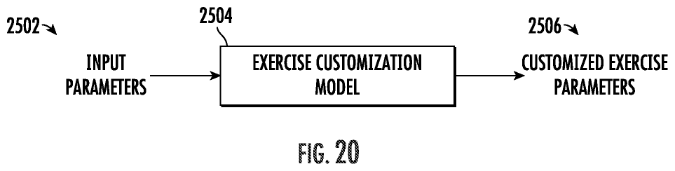

FIG. 20 illustrates an exercise customization model 2504 according to aspects of the present disclosure. The exercise customization model 2504 can be configured to receive input parameters 2502, and in response to receiving the input parameters 2502, output customized exercise parameters 2506. The output customized exercise parameters 2506 can include a frequency of exercise repetitions, a frequency of exercise sets, and/or a frequency of exercise sessions. As examples of such frequencies, the customized exercise parameters 2506 can include a number of repetitions per set, a number of sets per day, per week, per month, etc., a number of exercise sessions per day, per week, per month, etc., respective times during the day associated with each exercise set or exercise session, and/or respective days associated with each exercise set or exercise session. The customized exercise parameters 2506 can include rest times between sets, hold times for respective exercises, a pace of exercises within a set, and/or any other parameters associated with the user's customized training and/or treatment plan.

The input parameters 2502 can include each of the above-listed customized exercise parameters 2506. Additionally, the input parameters 2502 can include information about the user (e.g., Inco Types) and/or user feedback. For example, the user feedback can include an indication that the user would prefer that any of the above parameters should be increased, decreased, or maintained. Additional input parameters 2502 can include data describing user adherence and/or compliance with their plan. For instance, the input parameters 2502 can include metrics associated with a number of completed exercises (e.g., as compared with a number of prescribed exercises), times of day that the user is more likely to response to a prompt to perform an exercise, and/or days of the that the user is more likely to response to a prompt to perform an exercise.

As one example, the exercise customization model 2504 can output customized exercise parameters 2506 that include a reduced number of prescribed exercises at a particular day and/or time in response to input parameters 2502 that include or describe poor user compliance during particular days and/or times. As another example, the exercise customization model 2504 can output customized exercise parameters 2506 that include a reduced number of prescribed exercise sets of a particular exercise in response to user feedback requesting fewer of that particular exercise. In this example, the customized exercise parameters 2506 can include an increased number of a different exercise to compensate for the reduction in the particular exercise. As another example, the exercise customization model 2504 can output customized exercise parameters 2506 including increased sets, repetitions, or the like based on the user's progress, which can be determined user feedback, user compliance with the plan, a number of reported leakage events or the like. In some embodiments, the exercise customization model 2504 can be or include one or more machine-learned models 1010, for example, as described below with reference to FIG. 21. The machine-learned models can be or can be configured (e.g., trained) as various models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks.

In some embodiments, the exercise customization model 2504 can include or employ a variety of suitable algorithms and/or models, such as ladder algorithms, support vector machines, decision trees, ensemble models, k-nearest neighbors models, Bayesian networks, or other types of models including linear models and/or non-linear models.

FIG. 21 depicts a block diagram of an example computing system 100 that can perform incontinence logging according to example embodiments of the present disclosure. The system 100 can include a user computing device 102, a machine learning computing system 1030, and a digital application computing system 1130 that can be communicatively coupled over a network 1080.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 can include one or more processors 1012 and a memory 1214. The one or more processors 1012 can be any suitable processing device (e.g., a processor core, a microprocessor, an ASIC, a FPGA, a controller, a microcontroller, etc.) and can be one processor or a plurality of processors that are operatively connected. The memory 1214 can include one or more non-transitory computer-readable storage mediums, such as RAM, ROM, EEPROM, EPROM, flash memory devices, magnetic disks, etc., and combinations thereof. The memory 1214 can store data 1016 and instructions 1018 which are executed by the processor 1012 to cause the user computing device 102 to perform operations.

In some implementations, the user computing device 102 can store or include a display 1226 for providing visual content such as an interactive user interface 1228 and/or one or more machine-learned model(s) 1010. For example, a digital application implemented by the user computing device 102 can include or otherwise access the machine-learned model(s) 1010 and/or the instructions associated with the digital application computing system 1138 to determine suggested responses (e.g., using the machine-learned models(s) or other instructions associated with the digital application) and provide the suggested responses for view on an interactive user interface 1228 provided on the display 1226. For instance, some implementations may not include machine-learned model(s) 1010 and instead include instructions 1018, 1138 for providing suggested responses for view on the interactive user interface 1228 based on other conditions such as a logging history associated with a user account registered to the digital application.

In some implementations, instructions for providing the interactive user interface 1228 can be received from a digital application computing system 1130 (e.g., a server computing system and/or other computing system that is remote and/or distinct from the user computing device 1002) over network 1080, stored in the user computing device 102 memory 1214, and the used or otherwise implemented by the one or more processors 1012.

In some embodiments, the digital application computing system 1130 can be, include, or be connected with a physician computing system. The user can share exercise plans, logs, scorecards, and/or progress with his or her physician. For example, the user can export summaries, charts, raw data, or the like in a suitable data format to share with the doctor (e.g., digitally or by printing and sharing a hard copy). As another example, with the user's consent, the user's physician can access logged information, edit the user's exercise plan, or otherwise engage with the user and/or the user's experience with the system 100. The physician may have a portal for the system 100 to access and/or edit such data. As a further example, in some embodiments, the system 100 can include and/or interface directly with an electronic medical records (EMR) system. For instance, the digital application computing system 1130 can include one or more servers providing an electronic medical records system.

For some implementations, the instructions 1138 for implementing the digital application may be provided to a plurality of users. For instance, the instructions 1138 can be downloaded over a network 1080 such as the internet to execute the digital application on a plurality of user computing devices 102. The instructions 1138 can include methods for storing user data at the local device (e.g., the user computing device 102) and/or storing user data at a remote computing system (e.g., the digital application computing system 1130). As one example, the instructions 1138 can include providing for display on the user computing device an agreement to store data locally or transmit data for storage on the digital application computing system. In this manner, some implementations can include data privacy features by providing prompts to local devices using the digital application.

The instructions 1138 can also include methods for logging incontinence events, and/or methods for accessing incontinence support features such as a community that can be implemented as a public forum to registered users of the digital application and/or an avatar for supporting user interaction with the digital application. Aspects of the community and/or the avatar are provided in more detail in examples through the present disclosure.

In some implementations, the user computing device 102 can store instructions for providing an interactive user interface 1228 on a display 1226 (e.g., LCD, LED, or other screen capable of rendering visual content). For example, the user computing device 102 may include instructions for displaying content from applications via an application programming interface (API) associated with the digital application and or the user computing device 102.

For implementations that include one or more machine-learned models 1010, the machine-learned models 1010 can be or can be configured (e.g., trained) as various models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks.

In some implementations, the one or more machine-learned models 1010 can be received from a machine-learned computing system 1030 over network 1080, stored in the computing system memory 1214, and then used or otherwise implemented by the one or more processors 1012.

For certain implementations, the one or more machine-learned models 1040 can be included in or otherwise stored and implemented by the machine-learning computing system 1030 that communicates with the user computing device 102 according to a client-server relationship. One or more machine-learned models 1042 can be included in or otherwise stored and implemented by the digital application computing system 1034, which can communicate with the user computing device 102 according to a client-server relationship. For example, the machine-learned models 1040 can be implemented by the machine learning computing system 1030 and/or machine-learned models 1042 can be implemented by the digital application computing system 1034 as a portion of a web service. Thus, one or more machine-learned models 1010 can be stored and implemented at the user computing device 102; one or more models 1040 can be stored and implemented at the machine learning computing system 130; and/or one or more machine-learned models 1042 can be implemented by the digital application computing system 1034.

The user computing device 102 can also include one or more user input components that receive and/or detect user input (e.g., via the interactive user interface 1228, a microphone, camera, accelerometer, and/or other sensors). Example user inputs can include voice commands, gestures, and/or touch inputs directed to a touch-sensitive component (e.g., a touch-sensitive display screen or a touch pad) that is sensitive to the touch of a user input object (e.g., a finger or a stylus). The interactive use interface 1228 (e.g., including the touch-sensitive component) can serve to implement a virtual keyboard. Other example user input components include a microphone, a traditional keyboard, a mouse, or other means by which a user can provide user input.

The machine learning computing system 1030 can include one or more processors 1032 and a memory 1034. The one or more processors 1032 can be any suitable processing device (e.g., a processor core, a microprocessor, an ASIC, a FPGA, a controller, a microcontroller, etc.) and can be one processor or a plurality of processors that are operatively connected. The memory 1034 can include one or more non-transitory computer-readable storage mediums, such as RAM, ROM, EEPROM, EPROM, flash memory devices, magnetic disks, etc., and combinations thereof. The memory 1034 can store data 1036 and instructions 1038 which are executed by the processor 1032 to cause the machine learning computing system 130 to perform operations.

In some implementations, the machine learning computing system 1030 includes or is otherwise implemented by one or more server computing devices. In instances in which the machine learning computing system 1030 includes plural server computing devices, such server computing devices can operate according to sequential computing architectures, parallel computing architectures, or some combination thereof.

As described above, the machine learning computing system 1030 can store or otherwise include one or more machine-learned models 1040. For example, the models 1040 can be or can otherwise include various machine-learned models. Example machine-learned models include neural networks or other multi-layer non-linear models. Example neural networks include feed forward neural networks, deep neural networks, recurrent neural networks, and convolutional neural networks.

The user computing device 102, the machine learning computing system 1030, and/or digital application computing system can train the models 1010, 1040, 1042 via interaction with the model trainer 1050 that can include various algorithms for performing optimization of machine-learned models using supervised, unsupervised, and semi-supervised learning techniques. The machine learning computing system 1030 can also include training data 1052 that can be used to train machine-learned-learned models using the model trainer 1050. Some non-limiting examples of training or learning techniques can include, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 1060 can perform a number of generalization techniques (e.g., weight decays, dropouts, etc.) to improve the generalization capability of the models being trained.

In particular, the model trainer 1060 can train the machine-learned models 1010 and/or 1040 based on a set of training data 1062. As one example, the training data 1062 can include data collected private user logs that can be anonymized by associating the data with a unique user id. As another example, the training data 1062 can include responses to system notification such as prompts that request feedback based on user progress. In this manner, system notifications can be used as a form of labeling to create training datasets that include self-labeled data.

The model trainer 1060 can include computer logic utilized to provide desired functionality. The model trainer 1060 can be implemented in hardware, firmware, and/or software controlling a general purpose processor. For example, in some implementations, the model trainer 1060 includes program files stored on a storage device, loaded into a memory and executed by one or more processors. In other implementations, the model trainer 1060 includes one or more sets of computer-executable instructions that are stored in a tangible computer-readable storage medium such as RAM hard disk or optical or magnetic media.

It should be understood that statistical and/or algorithmics models can be used in place and/or in conjunction with one or more of the model(s) 1010, 1040, 1042. For example, K—means techniques can be leveraged for questioning analysis.

The computing system 100 can include one or more peripheral device(s) 1050, such as electrical stimulation devices (e.g., for applying electrical signals to muscles), electrical sensor devices (e.g., for detecting muscle contractions), cameras, microphones, accelerometers (e.g., pedometers), smartwatches, volatile organic compound sensors (e.g., configured to detect one more characteristics of a bodily fluid, such as urine or feces).

The network 1080 can be any type of communications network, such as a local area network (e.g., intranet), wide area network (e.g., Internet), or some combination thereof and can include any number of wired or wireless links. In general, communication over the network 1080 can be carried via any type of wired and/or wireless connection, using a wide variety of communication protocols (e.g., MQTT, TCP/IP, HTTP, SMTP, FTP), encodings or formats (e.g., HTML, XML), and/or protection schemes (e.g., TLS, VPN, secure HTTP, SSL). FIG. 21 illustrates one example computing system that can be used to implement the present disclosure. Other computing systems can be used as well. For example, in some implementations, the user computing device 102 can include the model trainer 1060 and the training dataset 1062. In such implementations, the machine-learned models 1010 can be both trained and used locally at the user computing device 102. In some of such implementations, the user computing device 102 can implement the model trainer 1060 to personalize the models 1010 based on user-specific data.

### Additional Disclosure

The technology discussed herein refers to servers, databases, software applications, and other computer-based systems, as well as actions taken, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computer-implemented method for logging incontinence information, the method comprising:
detecting, by one or more computing devices, a user input describing incontinence information;
updating, by the one or more computing devices and in response to detecting the user input, a private log based at least in part on the incontinence information;
determining, by the one or more computing devices and after updating the private log, a performance metric based at least in part on comparing the private log with a current plan; and
providing, by the one or more computing devices, a progress notification based at least in part on the performance metric.

2. The computer-implemented method of claim 1, further comprising:
displaying, by the one or more computing devices, a user interface comprising an avatar interface element;
detecting, by the one or more computing devices, a user input directed to the avatar interface element; and
in response to receiving the user interaction directed to the avatar interface element, one or more of creating a personal avatar and modifying the personal avatar.

3. The computer-implemented method of claim 1, further comprising:
displaying, by the one or more computing devices, a user interface comprising a community element;
detecting, by the one or more computing devices, a user input directed to the community element;
in response to receiving the user input directed to the community element, providing for display, by the one or more computing devices, data describing a public board comprising one or more forum interface elements for posting content to the public board.

4. The computer-implemented method of claim 3, further comprising:
providing, by the one or more computing devices, a reminder in response to detecting the user input directed to the community element, detecting the user input directed to an avatar interface element, or both.

5. The computer-implemented method of claim 1, further comprising:
generating, by the one or more computing devices, a set of one or more response options;
providing for display, by the one or more computing devices, data describing one or more response options; and
detecting, by the one or more computing devices, a user input directed to at least one of the one or more response options;
updating, by the one or more computing devices, the private log based at least in part on detecting the user input directed to the at least one of the one or more response options.

6. The computer-implemented method of claim 1, further comprising:
providing for display, by the one or more computing devices, data describing one or more learning plans each comprising one or more training exercises;
detecting, by the one or more computing devices, a user input describing a selection of at least one of the one or more learning plans; and
assigning, by the one or more computing devices, the at least one of the one or more learning plans as the current plan;
wherein each of the one or more training exercises comprise at least one of: a video, an audio, a game, and a text.

7. The computer implemented method of claim 1, further comprising:
providing for display, by the one or more computing devices, data describing a task;
detecting, by the one or more computing devices, a user input; and
updating, by the one or more computing devices and in response to detecting the input, a progress associated with the task.

8. The computer implemented method of claim 7, wherein the task comprises performing a Kegel.

9. The computer implemented method of claim 7, wherein the task comprises a bladder holding event.

10. The computer implemented method of 7, wherein detecting, by the one or more computing devices, the user input comprises detecting sensor data from at least one sensor.

11. The computer-implemented method of claim 1, further comprising:
determining, by the one or more computing devices, a self-progress metric based at least in part on comparing the private log on a first day to the private log on a second day that occurs after the first day; and
providing, by the one or more computing devices, a notification that describes the self-progress metric.

12. The computer-implemented method of claim 11, further comprising:
updating, by the one or more computing devices and based at least in part on the self progress metric at least one of the performance metric or a rewards account.

13. The computer-implemented method of claim 12, further comprising:
accessing, by the one or more computing devices, the rewards account to purchase an item; and
updating, by the one or more computing devices, the rewards account based on a cost associated with the item.

14. The computer-implemented method of claim 1, further comprising:
registering, by the one or more computing devices, a unique user id for a user.

15. The computer-implemented method of claim 1, further comprising:
generating, by the one or more computing devices, a global dataset by aggregating information included in respective private logs for a plurality of users; and
training, by the one or more computing devices, a global machine-learned model with an input comprising at least a portion of the global dataset, wherein the global machine-learned model is configured to receive an input comprising at least a portion of the private log associated with a unique user, and in response to receiving the input comprising the at least the portion of the private log associated with the unique user, provide an output that describes a suggested user action for the unique user.

16. The computer-implemented method of claim 15, further comprising:
providing for display, by the one or more computing devices and based at least in part on the output of the global machine-learned model, a prompt to assign one plan as the current plan.

17. The computer-implemented method of claim 1, further comprising:
generating, by the one or more computing devices, a personal dataset by aggregating information included in a private log associated with a user; and
training, by the one or more computing devices, a personal machine-learned model with an input describing at least a portion of the private log associated with the user, wherein the personal machine-learned model is configured to receive an input describing at least a portion of the private log associated with the user, and in response to receiving the input describing the at least the portion of the private log associated with user, provide an output that describes a suggested user action.

18. The computer-implemented method of claim 1, further comprising:
generating, by the one or more computing devices, a global dataset by aggregating information included in respective private logs for a plurality of users; and
training, by the one or more computing devices, a global machine-learned model with an input comprising the global dataset, wherein the global machine-learned model is configured to receive an input comprising at least a portion of the private log associated with a unique user, and in response to receiving the input comprising the at least the portion of the private log associated with the unique user, provide an output that describes a suggested user action for the unique user.

19. The computer-implemented method of claim 1, further comprising:
training, by the one or more computing devices, a global machine-learned model with an input comprising at least a portion of a global dataset that comprises aggregated incontinence information for a plurality of users; and
after training the global machine-learned model with the input comprising the at least the portion of the global dataset that comprises aggregated incontinence information for a plurality of users, further training, by the one or more computing devices, the global machine-learned model with incontinence information unique to a respective user to generate a personal machine-learned model for the respective user.

20. The computer-implemented method of claim 1, further comprising:
providing for display, by the one or more computing devices and based at least in part on the private log at least one of a question or an answer field; and
detecting, by the one or more computing devices, a user response directed to the answer field.

21. The computer-implemented method of claim 20, further comprising:
labeling, by the one or more computing devices, a machine-learning training dataset based at least in part on the user response.

21. (canceled)

22. The computer-implemented method of claim 1, further comprising:
providing, by the one or more computing devices, an instruction describing an incontinence exercise; and
detecting, by the one or more computing devices from a peripheral device, data describing performance of the incontinence exercise.

23. The computer-implemented method of claim 1, further comprising administering, by a peripheral device of the one or more computing devices, an incontinence treatment.

24. The computer-implemented method of claim 1, further comprising:
providing, by the one or more computing devices, an instruction describing an incontinence exercise;
detecting, by a camera of the one or more computing devices, data describing performance of the incontinence exercise; and
providing, by the one or more computing devices, feedback with respect to performance of the incontinence exercise.

25. The computer-implemented method of claim 1, further comprising:
inputting, by the one or more computing devices, one or more input parameters into an exercise customization model that is configured to receive the input parameters, and in response to receiving the input parameters, output customized exercise parameters.

26. The computer-implemented method of claim 25, wherein the customized exercise parameters comprise at least one of a frequency of sets, a frequency of exercise sessions, a frequency of exercise sessions, a number of repetitions per set, a rest time between sets, or a hold time for a given exercise.

27. A system for logging incontinence information, the system comprising:
one or more processors;
at least one tangible, non-transitory computer-readable medium that stores instructions that, when executed by the at least one processor, cause the one or more processors to perform operations, the operations comprising:
detecting a user input describing incontinence information;
updating, in response to detecting the user input, a private log based at least in part on the incontinence information;
determining, after updating the private log, a performance metric based at least in part on comparing the private log with a current plan; and
providing a progress notification based at least in part on the performance metric.

28. One or more non-transitory computer-readable media that collectively store instructions for a digital application for incontinence, the instructions for performing operations, the operations comprising:
detecting a user input describing incontinence information;
updating, in response to detecting the user input, a private log based at least in part on the incontinence information;
determining, after updating the private log, a performance metric based at least in part on comparing the private log with a current plan; and
providing a progress notification based at least in part on the performance metric.

29. The computer-implemented method of claim 1, wherein detecting the user input describing incontinence information comprises at least one of detecting a user touch input with respect to touch sensitive display, detecting audio, or detecting sensor data from one or more sensors
